(19) United States
(12) Patent Application Publication
(10) Pub. No.: US 2006/0018997 A1
(43) Pub. Date: Jan. 26, 2006

(54) Polypeptide

(76) Inventors: Karsten Matthias Kragh, Viby J (DK); Bo Spange Sorensen, Skanderborg (DK)

Correspondence Address: FROMMER LAWRENCE & HAUG, 745 FIFTH AVENUE- 10TH FL., NEW YORK, NY, 10151, US

(21) Appl. No.: 10/886,527

(22) Filed: Jul. 7, 2004

(51) Int. Cl. A21D 8/02; C12N 1/21

(52) U.S. Cl. 426/20; 435/252.34

(57) ABSTRACT

We describe a food additive comprising a PS4 variant polypeptide, in which the PS4 variant polypeptide is derivable from a parent polypeptide having non-maltogenic exoamylase activity, in which the PS4 variant polypeptide comprises an amino acid substitution at position 161 with reference to the position numbering of a Pseudomonas saccharophilia exoamylase sequence shown as SEQ ID NO: 1. The PS4 variant polypeptide may further comprise one or more further mutations at a position selected from the group consisting of: 121 and 223, preferably 121F, 121Y, 121W, 223E and 223K, more preferably G121F, G121Y, G121W, G223E and/or G223K, or the group consisting of: 134, 141, 157, 223, 307, 334, 33 and 34, preferably G134R, A141P, I157L, G223A, H307L, S334P, N33Y and D34N. In preferred embodiments, the PS4 variant polypeptide further comprises a mutation at position 87, preferably G87S, a mutation at position 178, preferably L178F, and/or a mutation at position 179, preferably A179T.

Reference is made to U.S. applications Ser. Nos. 60/485,413 and 60/485,616 filed Jul. 7, 2003, in which inventor Kragh is a co-inventor. Reference is also made to international applications filed Jul. 7, 2004 and designating the U.S. Ser. Nos. ______ (applicant: Genencor, attorney docket numbers GC806-PCT and GC807-PCT), in which inventor Kragh is a co-inventor. Reference is also made to U.S. application Ser. Nos. ______ to be assigned (attorney docket numbers GC806-US, GC807-US, and GC847-US) all of which were also filed Jul. 7, 2004, and in which inventor Kragh is a co-inventor.

Reference is additionally made to U.S. application Ser. No. 60/485,539 filed Jul. 7, 2003, in which inventor Kragh is a co-inventor. Reference is also made to international application filed Jul. 7, 2004 and designating the U.S. Ser. No. ______ (applicant: Danisco A/S, attorney docket number P016939WO), in which inventor Kragh is a co-inventor, and reference is also made to U.S. application Ser. Nos. ______ to be assigned (attorney docket numbers 674510-2007.1, 674510-2011 and 674510-2013), all of which were filed Jul. 7, 2004, and in which inventor Kragh is a co-inventor.

The foregoing applications, and each document cited or referenced in each of the present and foregoing applications, including during the prosecution of each of the foregoing applications (“application and article cited documents”), and any manufacturer's instructions or catalogues for any products cited or mentioned in each of the foregoing applications and articles and in any of the application and article cited documents, are hereby incorporated herein by reference. Furthermore, all documents cited in this text, and all documents cited or reference in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art.

### FIELD OF THE INVENTION

This invention relates to polypeptides, and nucleic acids encoding these, and their uses as non-maltogenic exoamylases in producing food products. In particular, the polypeptides are derived from polypeptides having non-maltogenic exoamylase activity, in particular, glucan 1,4-alpha-maltotetrahydrolase (EC 3.2.1.60) activity.

### SUMMARY OF THE INVENTION

According to a first aspect of the present invention, we provide a food additive comprising a PS4 variant polypeptide, in which the PS4 variant polypeptide is derivable from a parent polypeptide having non-maltogenic exoamylase activity, in which the PS4 variant polypeptide comprises an amino acid mutation at position 161 with reference to the position numbering of a Pseudomonas saccharophilia exoamylase sequence shown as SEQ ID NO: 1.

According to a second aspect of the invention, we provide a use of a PS4 variant polypeptide as set out in the first aspect of the invention as a food additive.

According to a third aspect of the invention, we provide a process for treating a starch comprising contacting the starch with a PS4 variant polypeptide as set out above and allowing the polypeptide to generate from the starch one or more linear products.

According to a fourth aspect of the invention, we provide use of a PS4 variant polypeptide as set out in the first aspect of the invention in preparing a food product.

According to a fifth aspect of the invention, we provide a process of preparing a food product comprising admixing a polypeptide as set out in the first aspect of the invention with a food ingredient.

According to a sixth aspect of the invention, we provide a process for making a bakery product comprising: (a) providing a starch medium; (b) adding to the starch medium a PS4 variant polypeptide as set out in the first aspect of the invention; and (c) applying heat to the starch medium during or after step (b) to produce a bakery product.

According to a seventh aspect of the invention, we provide a food product, dough product or a bakery product obtained by a process as described.

According to a eighth aspect of the invention, we provide an improver composition for a dough, in which the improver composition comprises a PS4 variant polypeptide as set out in the first aspect of the invention, and at least one further dough ingredient or dough additive.

According to a ninth aspect of the invention, we provide a composition comprising a flour and a PS4 variant polypeptide as set out in the first aspect of the invention.

According to a tenth aspect of the invention, we provide a use of a PS4 variant polypeptide as set out in the first aspect of the invention, in a dough product to retard or reduce staling, preferably detrimental retrogradation, of the dough product.

According to a eleventh aspect of the invention, we provide a combination of a PS4 variant polypeptide as set out above, together with Novamyl, or a variant, homologue, or mutants thereof which has maltogenic alpha-amylase activity.

According to a twelfth aspect of the invention, we provide a use of a Novamyl combination as described for an application as set out above.

According to a thirteenth aspect of the invention, we provide a food product produced by treatment with a combination as described.

### SEQUENCE LISTINGS

SEQ ID NO: 1 shows a PS4 reference sequence, derived from Pseudomonas saccharophila maltotetrahydrolase amino acid sequence.

SEQ ID NO: 2 shows a PSac-D34 sequence; Pseudomonas saccharophila maltotetrahydrolase amino acid sequence with 11 substitutions and deletion of the starch binding domain.

SEQ ID NO: 3 shows a PSac-D20 sequence; Pseudomonas saccharophila maltotetrahydrolase amino acid sequence with 13 substitutions and deletion of the starch binding domain.

SEQ ID NO: 4 shows a PSac-D14 sequence; Pseudomonas saccharophila maltotetrahydrolase amino acid sequence with 14 substitutions and deletion of the starch binding domain.

SEQ ID NO: 5 shows a Pseudomonas saccharophila Glucan 1,4-alpha-maltotetrahydrolase precursor (EC 3.2.1.60) (G4-amylase) (Maltotetraose-forming amylase) (Exo-maltotetraohydrolase) (Maltotetraose-forming exo-amylase). SWISS-PROT accession number P22963.

SEQ ID NO: 6 shows a P. saccharophila mta gene encoding maltotetraohydrolase (EC number=3.2.1.60). GenBank accession number X16732.

SEQ ID NO:7 shows a PS4 reference sequence, derived from Pseudomonas stutzeri maltotetrahydrolase amino acid sequence.

SEQ ID NO: 8 shows a PStu-D34 sequence; Pseudomonas stutzeri maltotetrahydrolase amino acid sequence with 9 substitutions.

SEQ ID NO: 9 shows a PStu-D20 sequence; Pseudomonas stutzeri maltotetrahydrolase amino acid sequence with 11 substitutions.

SEQ ID NO: 10 shows a PStu-D14 sequence; Pseudomonas stutzeri maltotetrahydrolase amino acid sequence with 12 substitutions.

SEQ ID NO: 11 shows a Pseudomonas stutzeri (Pseudomonas perfectomarina). Glucan 1,4-alpha-maltotetrahydrolase precursor (EC 3.2.1.60) (G4-amylase) (Maltotetraose-forming amylase) (Exo-maltotetraohydrolase) (Maltotetraose-forming exo-amylase). SWISS-PROT accession number P13507.

SEQ ID NO: 12 shows a P. stutzeri maltotetraose-forming amylase (amyP) gene, complete cds. GenBank accession number M24516.

SEQ ID NO: 13 shows a pMD55 sequence; Pseudomonas saccharophila maltotetrahydrolase amino acid sequence with 11 substitutions (G134R, A141P, I157L, G223A, H307L, S334P, N33Y, D34N, L178F, A179T and G121F).

SEQ ID NO: 13 shows a pMD55 sequence; Pseudomonas saccharophila maltotetrahydrolase amino acid sequence with 11 substitutions (G134R, A141P, I157L, G223A, H307L, S334P, N33Y, D34N, L178F, A179T and G121F) and deletion of the starch binding domain.

### DETAILED DESCRIPTION

In the following description and examples, unless the context dictates otherwise, dosages of PS4 variant polypeptides are given in parts per million (micrograms per gram) of flour. For example, “1 D34” as used in Table 2 indicates 1 part per million of pSac-D34 based on weight per weight. Preferably, enzyme quantities or amounts are determined based on activity assays as equivalents of pure enzyme protein measured with bovine serum albumin (BSA) as a standard, using the assay described in Bradford (1976, A rapid and sensitive method for the quantification of microgram quantities of protein utilizing the principle of protein-dye binding. Anal. Biochem. 72:248-254).

In describing the different PS4 variant polypeptide variants produced or which are contemplated to be encompassed by this document, the following nomenclature will be adopted for ease of reference:
(i) where the substitution includes a number and a letter, e.g., 141P, then this refers to [position according to the numbering system/substituted amino acid]. Accordingly, for example, the substitution of an amino acid to proline in position 141 is designated as 141P;
(ii) where the substitution includes a letter, a number and a letter, e.g., A141P, then this refers to [original amino acid/position according to the numbering system/substituted amino acid]. Accordingly, for example, the substitution of alanine with proline in position 141 is designated as A141P.

Where the relevant amino acid at a position can be substituted by any amino acid, this is designated by [position according to the numbering system/X], e.g., 161X.

Multiple mutations may be designated by being separated by slash marks “/”, e.g. A141P/G223A representing mutations in position 141 and 223 substituting alanine with proline and glycine with alanine respectively.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855, Lars-Inge Larsson “Immunocytochemistry: Theory and Practice”, CRC Press inc., Baca Raton, Fla., 1988, ISBN 0-8493-6078-1, John D. Pound (ed); “Immunochemical Protocols, vol 80”, in the series: “Methods in Molecular Biology”, Humana Press, Totowa, N.J., 1998, ISBN 0-89603-493-3, Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

PS4 Variants

We provide for compositions comprising polypeptides which are variants of polypeptides having non-maltogenic exoamylase activity, as well as uses of such variant polypeptides and the compositions. The compositions include the polypeptide variants together with at least one other component. In particular, we provide for food additives comprising the polypeptides.

Specifically, we provide for PS4 variant polypeptides with sequence alterations comprising amino acid substitutions in a non-maltogenic exoamylase sequence. The amino acid substitutions is at position 161, with reference to the position numbering of a Pseudomonas saccharophilia exoamylase sequence shown as SEQ ID NO: 1.

The amino acid substitution is preferably a change to 161A, more preferably a S161A mutation.

Combination Mutations

Further substitutions at one more positions such as 121 and 223 may also be included. Thus, in general the subject mutation at position 161 may advantageously be combined with a single additional mutation at one of positions 121 and 223, or two additional mutations at positions 121 and 223.

The position 121 substitution, where present, is preferably selected from the group consisting of: 121F, 121Y, 121W, 121H, 121A, 121M, 121G, 121S, 121T, 121D, 121E, 121L, 121K and 121V. Preferably, the position 121 substitution is 121F, 121Y or 121W.

The position 161 substitution, where present, is preferably 161A, more preferably S161A. Where position 161 is mutated, a further mutation at position 160 may also be present, preferably 160D, more preferably E160D.

The position 223 substitution, where present, is preferably selected from the group consisting of: 223K, 223E, 223V, 223R, 223A, 223P and 223D. More preferably, the 223 substitution is 223E or 223K.

In particularly preferred embodiments, the further substitution or substitutions are selected from the group consisting of: 121F, 121Y, 121W, 223E and 223K, preferably G121F, G121Y, G121W, G223E and/or G223K. Thus, the PS4 variant polypeptide may comprise any number of additional mutations selected from the above in addition to S161A.

In one embodiment, the PS4 variant polypeptide comprises at least two mutations at positions selected from the group consisting of: 121, 161; 161, 223, preferably: 121F/Y/W, 161A; 161A, 223E/K.

In a further embodiment, the PS4 variant polypeptide comprises at least three mutations at positions selected from the group consisting of: 121, 161 and 223. In a particularly preferred embodiment, the PS4 variant polypeptide comprises at least the three following substitutions 121F/Y/W, 161A, 223E/K. Other mutations may be included, as set out below.

Such variant polypeptides are referred to in this document as “PS4 variant polypeptides”. Nucleic acids encoding such variant polypeptides are also disclosed and will be referred to for convenience as “PS4 variant nucleic acids”. PS4 variant polypeptides and nucleic acids will be described in further detail below.

The “parent” sequences, i.e., the sequences on which the PS4 variant polypeptides and nucleic acids are based, preferably are polypeptides having non-maltogenic exoamylase activity. The terms “parent enzymes” and “parent polypeptides” should be interpreted accordingly, and taken to mean the enzymes and polypeptides on which the PS4 variant polypeptides are based. They are described in further detail below.

PS4 Variant Polypeptides

PS4 variant polypeptides and nucleic acids vary from their parent sequences by including a number of mutations. In other words, the sequence of the PS4 variant polypeptide or nucleic acid is different from that of its parent at a number of positions or residues. In preferred embodiments, the mutations comprise amino acid substitutions, that is, a change of one amino acid residue for another. Thus, the PS4 variant polypeptides comprise a number of changes in the nature of the amino acid residue at one or more positions of the parent sequence.

In particularly preferred embodiments, the parent sequences are non-maltogenic exoamylase enzymes, preferably bacterial non-maltogenic exoamylase enzymes. In highly preferred embodiments, the parent sequence comprises a glucan 1,4-alpha-maltotetrahydrolase (EC 3.2.1.60). Preferably, the parent sequence is derivable from Pseudomonas species, for example Pseudomonas saccharophilia or Pseudomonas stutzeri.

In some embodiments, the parent polypeptide comprises, or is homologous to, a wild type non-maltogenic exoamylase sequence, e.g., from Pseudomonas spp.

Thus, the parent polypeptide may comprise a Pseudomonas saccharophilia non-maltogenic exoamylase having a sequence shown as SEQ ID NO: 1. In other preferred embodiments, the parent polypeptide comprises a non-maltogenic exoamylase from Pseudomonas stutzeri having a sequence shown as SEQ ID NO: 11, or a Pseudomonas stutzeri non-maltogenic exoamylase having SWISS-PROT accession number P13507.

Proteins and nucleic acids related to, preferably having sequence or functional homology with Pseudomonas saccharophilia non-maltogenic exoamylase sequence shown as SEQ ID NO: 1 or a Pseudomonas stutzeri non-maltogenic exoamylase having a sequence shown as SEQ ID NO: 11 are referred to in this document as members of the “PS4 family”. Examples of “PS4 family” non-maltogenic exoamylase enzymes suitable for use in generating the PS4 variant polypeptides and nucleic acids are disclosed in further detail below.

In such embodiments where the parent polypeptide comprises a wild type sequence, the PS4 variant polypeptides will comprise a wild type sequence but with a mutation at position 161, preferably 161A, more preferably S161A.

In other, particularly preferred, embodiments, the parent sequences comprise already mutated PS4 sequences. Thus, where, as optionally set out above, for example, one or more other substitutions at positions 121 and 223 are included, these will also be present in the PS4 variant sequence. Furthermore, parent sequences comprising mutations at other positions, for example, any one or more of 134, 141, 157, 223, 307 and 334 may also be used. Optionally, these may include mutations at one or both of positions 33 and 34.

Thus, the parent sequence may comprise one or more mutations at positions selected from the group consisting of: 134, 141, 157, 223, 307, 334 and optionally 33 and 34, (and accordingly of course the PS4 variant polypeptides will also contain the relevant corresponding mutations).

We therefore disclose PS4 variant polypeptides comprising a mutation at position 161, preferably 161A more preferably S161A, and mutations at any one or more of positions 134, 141, 157, 223, 307, 334 and optionally 33 and 34. Mutations at 121 and 223 may of course also be included, as set out above.

In some embodiments, the parent polypeptides comprise substitutions arginine at position 134, proline at position 141 and proline at position 334, e.g., G134R, A141P and S334P.

In further preferred embodiments, the parent polypeptide further comprises a mutation at position 121. The parent polypeptide may further comprise a mutation at position 178. It may further comprise a mutation at position 179. It may yet further comprise a mutation at position 87. The respective particularly preferred substitutions are preferably 121D, more preferably G121D, preferably 178F, more preferably L178F, preferably 179T, more preferably A179T and preferably 87S, more preferably G87S.

The residues at these positions may be substituted by a number of residues, for example I157V or I157N or G223L or G223I or G223S or G223T or H307I or H307V or D34G or D34A or D34S or D34T or A179V. However, the parent polypeptides preferably comprise the substitutions I157L, G223A, H307L, L178F and A179T (optionally N33Y, D34N).

In a highly preferred embodiment, the PS4 variant polypeptides comprise a substitution at position 161 as well as one or more of the following substitutions: G134R, A141P, I157L, G223A, H307L, S334P, N33Y and D34N, together with one or both of L178F and A179T. The PS4 variant polypeptide may be derivable from a parent polypeptide having such substitutions.

Thus, in one embodiment, the PS4 variants are derivable from a Pseudomonas saccharophila non-maltogenic enzyme sequence comprising a sequence PSac-D34 (SEQ ID NO: 2).

In a further highly preferred embodiment, the PS4 variant polypeptides comprise a substitution at position 161 as well as one or more of the following substitutions: G134R, A141P, I157L, G223A, H307L, S334P, N33Y, D34N and G121D, together with one or both of L178F and A179T. The PS4 variant polypeptide may be derivable from a parent polypeptide having such substitutions.

Therefore, a PS4 variant may be based on a Pseudomonas saccharophila non-maltogenic parent enzyme sequence PSac-D20 (SEQ ID NO: 3).

In a yet further highly preferred embodiment, the PS4 variant polypeptides comprise a substitution at position 161 as well as one or more of the following substitutions: G134R, A141P, I157L, G223A, H307L, S334P, N33Y, D34N, G121D and G87S, together with one or both of L178F and A179T. The PS4 variant polypeptide may be derivable from a parent polypeptide having such substitutions.

Therefore, a PS4 variant may be based on a Pseudomonas saccharophila non-maltogenic parent enzyme sequence PSac-D14 (SEQ ID NO: 4).

In a yet another highly preferred embodiment, the PS4 variant polypeptides comprise a substitution at position 161 as well as one or more of the following substitutions: G134R, A141P, I157L, G223A, H307L, S334P, N33Y, D34N, G121F and G87S, together with one or both of L178F and A179T. The PS4 variant polypeptide may be derivable from a parent polypeptide having such substitutions.

Therefore, a PS4 variant may be based on a Pseudomonas saccharophila non-maltogenic parent enzyme sequence pMD55 (SEQ ID NO: 13).

In some embodiments, the PS4 variants are derived from a Pseudomonas stutzeri non-maltogenic enzyme sequence, preferably shown as SEQ ID NO: 7 below.

Accordingly, the PS4 variant polypeptide may be derivable from a sequence PStu-D34 (SEQ ID NO: 8). We further disclose PS4 variant polypeptides based on Pseudomonas stutzeri non-maltogenic enzyme sequence and including G121 and/or G87 substitutions. These may comprise the following substitutions: G134R, A141P, I157L, G223A, H307L, S334P, N33Y, D34N and G121D, together with one or both of L178F and A179T, as well as PS4 variant polypeptides comprising the following substitutions: G134R, A141P, I157L, G223A, H307L, S334P, N33Y, D34N, G121D and G87S, together with one or both of L178F and A179T.

Therefore, a PS4 variant polypeptide may be derived from a Pseudomonas stutzeri non-maltogenic enzyme parent sequence, which parent sequence may have a sequence PStu-D20 (SEQ ID NO: 9), comprising G121D, or a sequence PStu-D14 (SEQ ID NO: 10), further comprising G87S.

The PS4 variant polypeptides described in this document preferably retain the features of the parent polypeptides, and additionally preferably have additional beneficial properties, for example, enhanced activity or thermostability, or pH resistance, or any combination (preferably all). In particular, a PS4 variant polypeptide having a substitution at position 161 as described in this document preferably has an increased or enhanced exo-specificity and thermostability, as described in further detail below.

The PS4 substitution mutants described here may be used for any suitable purpose. They may preferably be used for purposes for which the parent enzyme is suitable. In particular, they may be used in any application for which exo-maltotetraohydrolase is used. In highly preferred embodiments, they have the added advantage of higher thermostability, or higher exoamylase activity or higher pH stability, or any combination, preferably higher exo-specificity and thermostability. Examples of suitable uses for the PS4 variant polypeptides and nucleic acids include food production, in particular baking, as well as production of foodstuffs; further examples are set out in detail below.

The PS4 variant polypeptides may comprise one or more further mutations in addition to position 161, and in addition to those set out above. There may be one, two, three, four, five, six, seven or more mutations preferably substitutions in addition to position 161, and/or in addition to those already set out. Other mutations, such as deletions, insertions, substitutions, transversions, transitions and inversions, at one or more other locations, may also be included. In addition, the PS4 variants need not have all the substitutions at the positions listed. Indeed, they may have one, two, three, four, or five substitutions missing, i.e., the wild type amino acid residue is present at such positions.

PS4 Variant Nucleic Acids

We also describe PS4 nucleic acids having sequences which correspond to or encode the alterations in the PS4 variant polypeptide sequences, for use in producing such polypeptides for the purposes described here.

The skilled person will be aware of the relationship between nucleic acid sequence and polypeptide sequence, in particular, the genetic code and the degeneracy of this code, and will be able to construct such PS4 nucleic acids without difficulty. For example, he will be aware that for each amino acid substitution in the PS4 variant polypeptide sequence, there may be one or more codons which encode the substitute amino acid. Accordingly, it will be evident that, depending on the degeneracy of the genetic code with respect to that particular amino acid residue, one or more PS4 nucleic acid sequences may be generated corresponding to that PS4 variant polypeptide sequence. Furthermore, where the PS4 variant polypeptide comprises more than one substitution, for example A141P/G223A, the corresponding PS4 nucleic acids may comprise pairwise combinations of the codons which encode respectively the two amino acid changes.

The PS4 variant nucleic acid sequences may be derivable from parent nucleic acids which encode any of the parent polypeptides described above. In particular, parent nucleic acids may comprise wild type sequences, e.g., SEQ ID NO: 6 or SEQ ID NO: 12. The PS4 variant nucleic acids may therefore comprise nucleic acids encoding wild type non-maltogenic exoamylases, but which encode another amino acid at position 161 instead of amino acid S.

They may also comprise wild type sequences with one or more mutations, e.g., which encode parent polypeptides having any of the substitutions G134R, A141P, I157L, G223A, H307L, S334P, N33Y, D34N, G121D and G87S, together with one or both of L178F and A179T.

Thus, for example, a PS4 nucleic acid sequence may be derivable from a parent sequence encoding a polypeptide having non-maltogenic exoamylase activity and comprising codons encoding amino acid substitutions at S161A as well as at least one of, preferably all, the following positions: G134, A141, I157, G223, H307, S334, N33 and D34, together with one or both of L178 and A179, with reference to the position numbering of a Pseudomonas saccharophilia exoamylase sequence shown as SEQ ID NO: 1.

We also describe a nucleic acid sequence derivable from a parent sequence, the parent sequence capable of encoding a non-maltogenic exoamylase, which nucleic acid sequence comprises a substitution at one or more residues such that the nucleic acid encodes S161A as well as one or more of the following mutations at the positions specified: G134, A141, I157, G223, H307, S334, N33 and D34, together with one or both of L178 and A179, with reference to the position numbering of a Pseudomonas saccharophilia exoamylase sequence shown as SEQ ID NO: 1.

It will be understood that nucleic acid sequences which are not identical to the particular PS4 variant nucleic acid sequences, but are related to these, will also be useful for the methods and compositions described here, such as a variant, homologue, derivative or fragment of a PS4 variant nucleic acid sequence, or a complement or a sequence capable of hybridising thereof. Unless the context dictates otherwise, the term “PS4 variant nucleic acid” should be taken to include each of these entities listed above.

Mutations in amino acid sequence and nucleic acid sequence may be made by any of a number of techniques, as known in the art. In particularly preferred embodiments, the mutations are introduced into parent sequences by means of PCR (polymerase chain reaction) using appropriate primers, as illustrated in the Examples. It is therefore possible to alter the sequence of a polypeptide by introducing amino acid substitutions comprising: G134, A141, I157, G223, H307, S334, N33 and D34, together with one or both of L178 and A179, into a parent polypeptide having non-maltogenic exoamylase activity, such as into a Pseudomonas saccharophilia or a Pseudomonas stutzeri exoamylase sequence at amino acid or nucleic acid level, as described. We describe a method in which the sequence of a non-maltogenic exoamylase is altered by altering the sequence of a nucleic acid which encodes the non-maltogenic exoamylase.

However, it will of course be appreciated that the PS4 variant polypeptide does not need in fact to be actually derived from a wild type polypeptide or nucleic acid sequence by, for example, step by step mutation. Rather, once the sequence of the PS4 variant polypeptide is established, the skilled person can easily make that sequence from the wild type with all the mutations, via means known in the art, for example, using appropriate oligonucleotide primers and PCR. In fact, the PS4 variant polypeptide can be made de novo with all its mutations, through, for example, peptide synthesis methodology.

In general, however, the PS4 variant polypeptides and/or nucleic acids are derived or derivable from a “precursor” sequence. The term “precursor” as used herein means an enzyme that precedes the enzyme which is modified according to the methods and compositions described here. Thus, the precursor may be an enzyme that is modified by mutagenesis as described elsewhere in this document. Likewise, the precursor may be a wild type enzyme, a variant wild type enzyme or an already mutated enzyme.

The PS4 variant polypeptides and nucleic acids may be produced by any means known in the art. Specifically, they may be expressed from expression systems, which may be in vitro or in vivo in nature. Specifically, we describe plasmids and expression vectors comprising PS4 nucleic acid sequences, preferably capable of expressing PS4 variant polypeptides. Cells and host cells which comprise and are preferably transformed with such PS4 nucleic acids, plasmids and vectors are also disclosed, and it should be made clear that these are also encompassed in this document.

In preferred embodiments, the PS4 variant polypeptide sequence is used as a food additive in an isolated form. The term “isolated” means that the sequence is at least substantially free from at least one other component with which the sequence is naturally associated in nature and as found in nature. In one aspect, preferably the sequence is in a purified form. The term “purified” means that the sequence is in a relatively pure state—e.g. at least about 90% pure, or at least about 95% pure or at least about 98% pure.

Position Numbering

All positions referred to in the present document by numbering refer to the numbering of a Pseudomonas saccharophilia exoamylase reference sequence shown below (SEQ ID NO: 1):

| | |
|---|---|
| 1 | DQAGKSPAGV RYHGGDEIIL QGFHWNVVRE APNDWYNILR QQASTIAADG FSAIWMPVPW |
| 61 | RDFSSWTD [symbol] G KSGGGEGYFW HDFNKNGRYG SDAQLRQAAG ALGGAGVKVL YDVVPNHMNR |
| 121 | GYPDKEINLP AGQGFWRNDC [symbol] DPGNYPNDC DDGDRFIGGE SDLNTGHPQI YGMFRDELAN |
| 181 | LRSGYGAGGF RFDFVRGYAP ERVDSWMSDS ADSSFCVGEL WK [symbol] PSEYPSW DWRNTASWQQ |
| 241 | IIKDWSDRAK CPVFDFALKE RMQNGSV [symbol] DW KHGLNGNPDP RWREVAVTFV DNHDTGYSPG |
| 301 | QNGGQHHWAL QD [symbol] LIRQAYA YILTSPGTPV VYWSHMYDWG YGDFIRQLIQ VRRTAGVRAD |
| 361 | SAISFHSGYS GLVATVSGSQ QTLVVALNSD LANPGQVA [symbol] SFSEAVNASN GQVRVWRSGS |
| 421 | GDGGGNDGGE GGLVNVNFRC DNGVTQMGDS VYAVGNVSQL GNWSPASAVR LTDTSSYPTW |
| 481 | KGSIALPDGQ NVEWKCLIRN EADATLVRQW QSGGNNQVQA AAGASTSGSF |

The reference sequence is derived from the Pseudomonas saccharophilia sequence having SWISS-PROT accession number P22963, but without the signal sequence MSHILRAAVLAAVLLPFPALA.

In the context of the present description a specific numbering of amino acid residue positions in PS4 exoamylase enzymes is employed. In this respect, by alignment of the amino acid sequences of various known exoamylases it is possible to unambiguously allot a exoamylase amino acid position number to any amino acid residue position in any exoamylase enzyme, the amino acid sequence of which is known. Using this numbering system originating from for example the amino acid sequence of the exoamylase obtained from Pseudomonas saccharophilia, aligned with amino acid sequences of a number of other known exoamylase, it is possible to indicate the position of an amino acid residue in a exoamylase unambiguously.

Therefore, the numbering system, even though it may use a specific sequence as a base reference point, is also applicable to all relevant homologous sequences. For example, the position numbering may be applied to homologous sequences from other Pseudomonas species, or homologous sequences from other bacteria. Preferably, such homologous have 60% or greater homology, for example 70% or more, 80% or more, 90% or more or 95% or more homology, with the reference sequence SEQ ID NO: 1 above, or the sequences having SWISS-PROT accession numbers P22963 or P13507, preferably with all these sequences. Sequence homology between proteins may be ascertained using well known alignment programs and hybridisation techniques described herein. Such homologous sequences, as well as the functional equivalents described below, will be referred to in this document as the “PS4 Family”.

Furthermore, and as noted above, the numbering system used in this document makes reference to a reference sequence SEQ ID NO: 1, which is derived from the Pseudomonas saccharophilia sequence having SWISS-PROT accession number P22963, but without the signal sequence MSHILRAAVLAAVLLPFPALA. This signal sequence is located N terminal of the reference sequence and consists of 21 amino acid residues. Accordingly, it will be trivial to identify the particular residues to be mutated or substituted in corresponding sequences comprising the signal sequence, or indeed, corresponding sequences comprising any other N- or C-terminal extensions or deletions. For example, the sequence of Pseudomonas saccharophilia non-maltogenic exoamylase having SWISS-PROT accession number P22963 or a Pseudomonas stutzeri non-maltogenic exoamylase having SWISS-PROT accession number P13507.

Parent Enzyme/Polypeptide

The PS4 variant polypeptides are derived from, or are variants of, another sequence, known as a “parent enzyme”, a “parent polypeptide” or a “parent sequence”.

The term “parent enzyme” as used in this document means the enzyme that has a close, preferably the closest, chemical structure to the resultant variant, i.e., the PS4 variant polypeptide or nucleic acid. The parent enzyme may be a precursor enzyme (i.e. the enzyme that is actually mutated) or it may be prepared de novo. The parent enzyme may be a wild type enzyme, or it may be a wild type enzyme comprising one or more mutations.

The term “precursor” as used herein means an enzyme that precedes the enzyme which is modified to produce the enzyme. Thus, the precursor may be an enzyme that is modified by mutagenesis. Likewise, the precursor may be a wild type enzyme, a variant wild type enzyme or an already mutated enzyme.

The term “wild type” is a term of the art understood by skilled persons and means a phenotype that is characteristic of most of the members of a species occurring naturally and contrasting with the phenotype of a mutant. Thus, in the present context, the wild type enzyme is a form of the enzyme naturally found in most members of the relevant species. Generally, the relevant wild type enzyme in relation to the variant polypeptides described here is the most closely related corresponding wild type enzyme in terms of sequence homology. However, where a particular wild type sequence has been used as the basis for producing a variant PS4 polypeptide as described here, this will be the corresponding wild type sequence regardless of the existence of another wild type sequence that is more closely related in terms of amino acid sequence homology.

The parent enzyme is preferably a polypeptide which preferably exhibits non-maltogenic exoamylase activity. Preferably, the parent enzyme is a non-maltogenic exoamylase itself. For example, the parent enzyme may be a Pseudomonas saccharophila non-maltogenic exoamylase, such as a polypeptide having SWISS-PROT accession number P22963, or a Pseudomonas stutzeri non-maltogenic exoamylase, such as a polypeptide having SWISS-PROT accession number P13507.

Other members of the PS4 family may be used as parent enzymes; such “PS4 family members” will generally be similar to, homologous to, or functionally equivalent to either of these two enzymes, and may be identified by standard methods, such as hybridisation screening of a suitable library using probes, or by genome sequence analysis.

In particular, functional equivalents of either of these two enzymes, as well as other members of the “PS4 family” may also be used as starting points or parent polypeptides for the generation of PS4 variant polypeptides as described here.

A “functional equivalent” of a protein means something that shares one or more, preferably substantially all, of the functions of that protein. Preferably, such functions are biological functions, preferably enzymatic functions, such as amylase activity, preferably non-maltogenic exoamylase activity.

The term “functional equivalent” in relation to a parent enzyme being a Pseudomonas saccharophila non-maltogenic exoamylase, such as a polypeptide having SWISS-PROT accession number P22963, or a Pseudomonas stutzeri non-maltogenic exoamylase, such as a polypeptide having SWISS-PROT accession number P13507 means that the functional equivalent could be obtained from other sources. The functionally equivalent enzyme may have a different amino acid sequence but will have non-maltogenic exoamylase activity.

In highly preferred embodiments, the functional equivalent will have sequence homology to either of the Pseudomonas saccharophila and Pseudomonas stutzeri non-maltogenic exoamylases mentioned above, preferably both. The functional equivalent may also have sequence homology with any of the sequences set out as SEQ ID NOs: 1 to 12, preferably SEQ ID NO: 1 or SEQ ID NO: 7 or both. Sequence homology between such sequences is preferably at least 60%, preferably 65% or more, preferably 75% or more, preferably 80% or more, preferably 85% or more, preferably 90% or more, preferably 95% or more. Such sequence homologies may be generated by any of a number of computer programs known in the art, for example BLAST or FASTA, etc. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

In other embodiments, the functional equivalents will be capable of specifically hybridising to any of the sequences set out above. Methods of determining whether one sequence is capable of hybridising to another are known in the art, and are for example described in Sambrook, et al (supra) and Ausubel, F. M. et al. (supra). In highly preferred embodiments, the functional equivalents will be capable of hybridising under stringent conditions, e.g. 65° C. and 0.1×SSC {1×SSC=0.15 M NaCl, 0.015 M Na₃ Citrate pH 7.0}.

For example, functional equivalents which have sequence homology to Pseudomonas saccharophila and Pseudomonas stutzeri non-maltogenic exoamylases are suitable for use as parent enzymes. Such sequences may differ from the Pseudomonas saccharophila sequence at any one or more positions. Furthermore, non-maltogenic exoamylases from other strains of Pseudomonas spp, such as ATCC17686, may also be used as a parent polypeptide. The PS4 variant polypeptide residues may be inserted into any of these parent sequences to generate the variant PS4 polypeptide sequences.

It will be understood that where it is desired for PS4 variant polypeptides to additionally comprise one or more mutations corresponding to, for example, 121F, 121Y, 121W, 223E and 223K, and/or G134R, A141P, I157L, G223A, H307L, S334P, N33Y and D34N, together with one or both of L178F and A179T, corresponding mutations may be made in the nucleic acid sequences of the functional equivalents of Pseudomonas spp non-maltogenic exoamylase, as well as other members of the “PS4 family”, in order that they may be used as starting points or parent polypeptides for the generation of PS4 variant polypeptides as described here.

Specifically, the polypeptides disclosed in U.S. applications Ser. Nos. 60/485,413 and 60/485,616 (to be assigned, attorney docket numbers GC806P and GC807P), in which inventor Kragh is a co-inventor, as well as those disclosed in concurrently filed PCT application designating the U.S. Ser. Nos. ______ (applicant: Genencor GC806-PCT and GC807-PCT), in which inventor Kragh is a co-inventor, are to be included within the term “PS4 variant polypeptides”. Such polypeptides are suitable for use in the applications described herein, in particular, as food additives, to treat starch as described, to prepare a food product, to make a bakery product, for the formulation of improver compositions, for the formulation of combinations, etc.

Modification of Parent Sequences

The parent enzymes may be modified at the amino acid level or the nucleic acid level to generate the PS4 variant sequences described here. Therefore, we provide for the generation of PS4 variant polypeptides by introducing one or more corresponding codon changes in the nucleotide sequence encoding a non-maltogenic exoamylase polypeptide.

The nucleic acid numbering should preferably be with reference to the position numbering of a Pseudomonas saccharophilia exoamylase nucleotide sequence shown as SEQ ID NO: 6. Alternatively, or in addition, reference may be made to the sequence with GenBank accession number X16732. In preferred embodiments, the nucleic acid numbering should be with reference to the nucleotide sequence shown as SEQ ID NO: 6. However, as with amino acid residue numbering, the residue numbering of this sequence is to be used only for reference purposes only. In particular, it will be appreciated that the above codon changes can be made in any PS4 family nucleic acid sequence. For example, sequence changes can be made to a Pseudomonas saccharophila or a Pseudomonas stutzeri non-maltogenic exoamylase nucleic acid sequence (e.g., X16732, SEQ ID NO: 6 or M24516, SEQ ID NO: 12).

The parent enzyme may comprise the “complete” enzyme, i.e., in its entire length as it occurs in nature (or as mutated), or it may comprise a truncated form thereof. The PS4 variant derived from such may accordingly be so truncated, or be “full-length”. The truncation may be at the N-terminal end, or the C-terminal end, preferably the C-terminal end. The parent enzyme or PS4 variant may lack one or more portions, such as sub-sequences, signal sequences, domains or moieties, whether active or not etc. For example, the parent enzyme or the PS4 variant polypeptide may lack a signal sequence, as described above. Alternatively, or in addition, the parent enzyme or the PS4 variant may lack one or more catalytic or binding domains.

In highly preferred embodiments, the parent enzyme or PS4 variant may lack one or more of the domains present in non-maltogenic exoamylases, such as the starch binding domain. For example, the PS4 polypeptides may have only sequence up to position 429, relative to the numbering of a Pseudomonas saccharophilia non-maltogenic exoamylase shown as SEQ ID NO: 1. It is to be noted that this is the case for the PS4 variants pSac-d34, pSac-D20 and pSac-D14.

Amylase

The PS4 variant polypeptides generally comprise amylase activity.

The term “amylase” is used in its normal sense—e.g. an enzyme that is inter alia capable of catalysing the degradation of starch. In particular they are hydrolases which are capable of cleaving α-D-(1→4) O-glycosidic linkages in starch.

Amylases are starch-degrading enzymes, classified as hydrolases, which cleave α-D-(1→4) O-glycosidic linkages in starch. Generally, α-amylases (E.C. 3.2.1.1, α-D-(1→4)-glucan glucanohydrolase) are defined as endo-acting enzymes cleaving α-D-(1→4) O-glycosidic linkages within the starch molecule in a random fashion. In contrast, the exo-acting amylolytic enzymes, such as β-amylases (E.C. 3.2.1.2, α-D-(1→4)-glucan maltohydrolase), and some product-specific amylases like maltogenic alpha-amylase (E.C. 3.2.1.133) cleave the starch molecule from the non-reducing end of the substrate. β-Amylases, α-glucosidases (E.C. 3.2.1.20, α-D-glucoside glucohydrolase), glucoamylase (E.C. 3.2.1.3, α-D-(1→4)-glucan glucohydrolase), and product-specific amylases can produce malto-oligosaccharides of a specific length from starch.

Non-Maltogenic Exoamylase

The PS4 variant polypeptides described in this document are derived from (or variants of) polypeptides which preferably exhibit non-maltogenic exoamylase activity. Preferably, these parent enzymes are non-maltogenic exoamylases themselves. The PS4 variant polypeptides themselves in highly preferred embodiments also exhibit non-maltogenic exoamylase activity.

In highly preferred embodiments, the term “non-maltogenic exoamylase enzyme” as used in this document should be taken to mean that the enzyme does not initially degrade starch to substantial amounts of maltose as analysed in accordance with the product determination procedure as described in this document.

In highly preferred embodiments, the non-maltogenic exoamylase comprises an exo-maltotetraohydrolase. Exo-maltotetraohydrolase (E.C.3.2.1.60) is more formally known as glucan 1,4-alpha-maltotetrahydrolase. This enzyme hydrolyses 1,4-alpha-D-glucosidic linkages in amylaceous polysaccharides so as to remove successive maltotetraose residues from the non-reducing chain ends.

Non-maltogenic exoamylases are described in detail in U.S. Pat. No. 6,667,065, hereby incorporated by reference.

Assays for Non-Maltogenic Exoamylase Activity

The following system is used to characterize polypeptides having non-maltogenic exoamylase activity which are suitable for use according to the methods and compositions described here. This system may for example be used to characterise the PS4 parent or variant polypeptides described here.

By way of initial background information, waxy maize amylopectin (obtainable as WAXILYS 200 from Roquette, France) is a starch with a very high amylopectin content (above 90%). 20 mg/ml of waxy maize starch is boiled for 3 min. in a buffer of 50 mM MES (2-(N-morpholino)ethanesulfonic acid), 2 mM calcium chloride, pH 6.0 and subsequently incubated at 50° C. and used within half an hour.

One unit of the non-maltogenic exoamylase is defined as the amount of enzyme which releases hydrolysis products equivalent to 1 μmol of reducing sugar per min. when incubated at 50 degrees C. in a test tube with 4 ml of 10 mg/ml waxy maize starch in 50 mM MES, 2 mM calcium chloride, pH 6.0 prepared as described above. Reducing sugars are measured using maltose as standard and using the dinitrosalicylic acid method of Bernfeld, Methods Enzymol., (1954), 1, 149-158 or another method known in the art for quantifying reducing sugars.

The hydrolysis product pattern of the non-maltogenic exoamylase is determined by incubating 0.7 units of non-maltogenic exoamylase for 15 or 300 min. at 50° C. in a test tube with 4 ml of 10 mg/ml waxy maize starch in the buffer prepared as described above. The reaction is stopped by immersing the test tube for 3 min. in a boiling water bath.

The hydrolysis products are analyzed and quantified by anion exchange HPLC using a Dionex PA 100 column with sodium acetate, sodium hydroxide and water as eluents, with pulsed amperometric detection and with known linear maltooligo-saccharides of from glucose to maltoheptaose as standards. The response factor used for maltooctaose to maltodecaose is the response factor found for maltoheptaose.

Preferably, the PS4 variant polypeptides have non-maltogenic exoamylase activity such that if an amount of 0.7 units of said non-maltogenic exoamylase were to incubated for 15 minutes at a temperature of 50° C. at pH 6.0 in 4 ml of an aqueous solution of 10 mg preboiled waxy maize starch per ml buffered solution containing 50 mM 2-(N-morpholino)ethane sulfonic acid and 2 mM calcium chloride then the enzyme would yield hydrolysis product(s) that would consist of one or more linear malto-oligosaccharides of from two to ten D-glucopyranosyl units and optionally glucose; such that at least 60%, preferably at least 70%, more preferably at least 80% and most preferably at least 85% by weight of the said hydrolysis products would consist of linear maltooligosaccharides of from three to ten D-glucopyranosyl units, preferably of linear maltooligosaccharides consisting of from four to eight D-glucopyranosyl units.

For ease of reference, and for the present purposes, the feature of incubating an amount of 0.7 units of the non-maltogenic exoamylase for 15 minutes at a temperature of 50° C. at pH 6.0 in 4 ml of an aqueous solution of 10 mg preboiled waxy maize starch per ml buffered solution containing 50 mM 2-(N-morpholino)ethane sulfonic acid and 2 mM calcium chloride, may be referred to as the “Waxy Maize Starch Incubation Test”.

Thus, alternatively expressed, preferred PS4 variant polypeptides which are non-maltogenic exoamylases are characterised as having the ability in the waxy maize starch incubation test to yield hydrolysis product(s) that would consist of one or more linear malto-oligosaccharides of from two to ten D-glucopyranosyl units and optionally glucose; such that at least 60%, preferably at least 70%, more preferably at least 80% and most preferably at least 85% by weight of the said hydrolysis product(s) would consist of linear maltooligosaccharides of from three to ten D-glucopyranosyl units, preferably of linear maltooligosaccharides consisting of from four to eight D-glucopyranosyl units.

The hydrolysis products in the waxy maize starch incubation test may include one or more linear malto-oligosaccharides of from two to ten D-glucopyranosyl units and optionally glucose. The hydrolysis products in the waxy maize starch incubation test may also include other hydrolytic products. Nevertheless, the % weight amounts of linear maltooligosaccharides of from three to ten D-glucopyranosyl units are based on the amount of the hydrolysis product that consists of one or more linear malto-oligosaccharides of from two to ten D-glucopyranosyl units and optionally glucose. In other words, the % weight amounts of linear maltooligosaccharides of from three to ten D-glucopyranosyl units are not based on the amount of hydrolysis products other than one or more linear malto-oligosaccharides of from two to ten D-glucopyranosyl units and glucose.

The hydrolysis products can be analysed by any suitable means. For example, the hydrolysis products may be analysed by anion exchange HPLC using a Dionex PA 100 column with pulsed amperometric detection and with, for example, known linear maltooligosaccharides of from glucose to maltoheptaose as standards.

For ease of reference, and for the present purposes, the feature of analysing the hydrolysis product(s) using anion exchange HPLC using a Dionex PA 100 column with pulsed amperometric detection and with known linear maltooligosaccharides of from glucose to maltoheptaose used as standards, can be referred to as “analysing by anion exchange”. Of course, and as just indicated, other analytical techniques would suffice, as well as other specific anion exchange techniques.

Thus, alternatively expressed, a preferred PS4 variant polypeptide is one which has non-maltogenic exoamylase such that it has the ability in a waxy maize starch incubation test to yield hydrolysis product(s) that would consist of one or more linear malto-oligosaccharides of from two to ten D-glucopyranosyl units and optionally glucose, said hydrolysis products being capable of being analysed by anion exchange; such that at least 60%, preferably at least 70%, more preferably at least 80% and most preferably at least 85% by weight of the said hydrolysis product(s) would consist of linear maltooligosaccharides of from three to ten D-glucopyranosyl units, preferably of linear maltooligosaccharides consisting of from four to eight D-glucopyranosyl units.

As used herein, the term “linear malto-oligosaccharide” is used in the normal sense as meaning 2-10 units of α-D-glucopyranose linked by an α-(1→4) bond.

In highly preferred embodiments, the PS4 polypeptides described here have improved exoamylase activity, preferably non-maltogenic exoamylase activity, when compared to the parent polypeptide, preferably when tested under the same conditions. In particular, in highly preferred embodiments, the PS4 variant polypeptides have 10% or more, preferably 20% or more, preferably 50% or more, exoamylase activity compared to their parents, preferably when measured in a waxy maize starch test.

The hydrolysis products can be analysed by any suitable means. For example, the hydrolysis products may be analysed by anion exchange HPLC using a Dionex PA 100 column with pulsed amperometric detection and with, for example, known linear maltooligosaccharides of from glucose to maltoheptaose as standards.

As used herein, the term “linear malto-oligosaccharide” is used in the normal sense as meaning 2-20 units of α-D-glucopyranose linked by an α-(1→4) bond.

Improved Properties

The PS4 variants described here preferably have improved properties when compared to their parent enzymes, such as any one or more of improved thermostability, improved pH stability, or improved exo-specificity. In particular, the PS4 variant polypeptides having mutations at position 161, preferably 161A, more preferably S161A, have increased exo-specificity and thermostability.

Without wishing to be bound by any particular theory, we believe that the mutations at the particular positions have individual and cumulative effects on the properties of a polypeptide comprising such mutations. Thus, for example, we believe that positions 134, 141, 157, 223, 334, 33 and 34, as well as positions 178 or 179, or both influence the thermostability of PS4 polypeptides comprising such changes. Particularly, and preferably, positive or beneficial effects reside in these positions, particular in the substitutions: 134R, 141P, 157L, 223A, 307L, 334P, 33Y and 34N, 178F and 179T where present.

On the other hand, we believe that positions 307, as well as position 121 have effects (preferably positive effects) on the exo-specificity of a PS4 polypeptide.

Thermostability and pH Stability

Preferably, the PS4 variant polypeptide is thermostable; preferably, it has higher thermostability than its parent enzyme.

In wheat and other cereals the external side chains in amylopectin are in the range of DP 12-19. Thus, enzymatic hydrolysis of the amylopectin side chains, for example, by PS4 variant polypeptides as described having non-maltogenic exoamylase activity, can markedly reduce their crystallisation tendencies.

Starch in wheat and other cereals used for baking purposes is present in the form of starch granules which generally are resistant to enzymatic attack by amylases. Thus starch modification is mainly limited to damaged starch and is progressing very slowly during dough processing and initial baking until gelatinisation starts at about 60 C. As a consequence hereof only amylases with a high degree of thermostability are able to modify starch efficiently during baking. And generally the efficiency of amylases is increased with increasing thermostability. That is because the more thermostable the enzyme is the longer time it can be active during baking and thus the more antistaling effect it will provide.

Accordingly, the use of PS4 variant polypeptides as described here when added to the starch at any stage of its processing into a food product, e.g., before during or after baking into bread can retard or impede or slow down the retrogradation. Such use is described in further detail below.

As used herein the term ‘thermostable’ relates to the ability of the enzyme to retain activity after exposure to elevated temperatures. Preferably, the PS4 variant polypeptide is capable of degrading starch at temperatures of from about 55° C. to about 80° C. or more. Suitably, the enzyme retains its activity after exposure to temperatures of up to about 95° C.

The thermostability of an enzyme such as a non-maltogenic exoamylase is measured by its half life. Thus, the PS4 variant polypeptides described here have half lives extended relative to the parent enzyme by preferably 10%, 20%, 30%, 40%, 50%, 60%, 70%, 80%, 90%, 100%, 200% or more, preferably at elevated temperatures of from 55° C. to about 95° C. or more, preferably at about 80° C.

As used here, the half life (t½) is the time (in minutes) during which half the enzyme activity is inactivated under defined heat conditions. In preferred embodiments, the half life is assayed at 80 degrees C. Preferably, the sample is heated for 1-10 minutes at 80° C. or higher. The half life value is then calculated by measuring the residual amylase activity, by any of the methods described here. Preferably, a half life assay is conducted as described in more detail in the Examples.

Preferably, the PS4 variants described here are active during baking and hydrolyse starch during and after the gelatinization of the starch granules which starts at temperatures of about 55° C. The more thermostable the non-maltogenic exoamylase is the longer time it can be active and thus the more antistaling effect it will provide. However, during baking above temperatures of about 85° C., enzyme inactivation can take place. If this happens, the non-maltogenic exoamylase may be gradually inactivated so that there is substantially no activity after the baking process in the final bread. Therefore preferentially the non-maltogenic exoamylases suitable for use as described have an optimum temperature above 50° C. and below 98° C.

The thermostability of the PS4 variants described here can be improved by using protein engineering to become more thermostable and thus better suited for the uses described here; we therefore encompass the use of PS4 variants modified to become more thermostable by protein engineering.

Preferably, the PS4 variant polypeptide is pH stable; more preferably, it has a higher pH stability than its cognate parent polypeptide. As used herein the term ‘pH stable’ relates to the ability of the enzyme to retain activity over a wide range of pHs. Preferably, the PS4 variant polypeptide is capable of degrading starch at a pH of from about 5 to about 10.5. In one embodiment, the degree of pH stability may be assayed by measuring the half life of the enzyme in specific pH conditions. In another embodiment, the degree of pH stability may be assayed by measuring the activity or specific activity of the enzyme in specific pH conditions. The specific pH conditions may be any pH from pH5 to pH10.5.

Thus, the PS4 variant polypeptide may have a longer half life, or a higher activity (depending on the assay) when compared to the parent polypeptide under identical conditions. The PS4 variant polypeptides may have 10%, 20%, 30%, 40%, 50%, 60%, 70%, 80%, 90%, 100%, 200% or longer half life when compared to their parent polypeptides under identical pH conditions. Alternatively, or in addition, they may have such higher activity when compared to the parent polypeptide under identical pH conditions.

Exo-Specificity

It is known that some non-maltogenic exoamylases can have some degree of endoamylase activity. In some cases, this type of activity may need to be reduced or eliminated since endoamylase activity can possibly negatively effect the quality of the final bread product by producing a sticky or gummy crumb due to the accumulation of branched dextrins.

Exo-specificity can usefully be measured by determining the ratio of total amylase activity to the total endoamylase activity. This ratio is referred to in this document as a “Exo-specificity index”. In preferred embodiments, an enzyme is considered an exoamylase if it has a exo-specificity index of 20 or more, i.e., its total amylase activity (including exo-amylase activity) is 20 times or more greater than its endoamylase activity. In highly preferred embodiments, the exo-specificity index of exoamylases is 30 or more, 40 or more, 50 or more, 60 or more, 70 or more, 80 or more, 90 or more, or 100 or more. In highly preferred embodiments, the exo-specificity index is 150 or more, 200 or more, 300 or more, 400 or more, 500 or more or 600 or more.

The total amylase activity and the endoamylase activity may be measured by any means known in the art. For example, the total amylase activity may be measured by assaying the total number of reducing ends released from a starch substrate. Alternatively the use of a Betamyl assay is described in further detail in the Examples, and for convenience, amylase activity as assayed in the Examples is described in terms of “Betamyl Units” in the Tables and Figures.

Endoamylase activity may be assayed by use of a Phadebas Kit (Pharmacia and Upjohn). This makes use of a blue labelled crosslinked starch (labelled with an azo dye); only internal cuts in the starch molecule release label, while external cuts do not do so. Release of dye may be measured by spectrophotometry. Accordingly, the Phadebas Kit measures endoamylase activity, and for convenience, the results of such an assay (described in the Examples) are referred to in this document as “Phadebas units”.

In a highly preferred embodiment, therefore, the exo-specificity index is expressed in terms of Betamyl Units/Phadebas Units, also referred to as “B/Phad”.

Exo-specificity may also be assayed according to the methods described in the prior art, for example, in our International Patent Publication Number WO99/50399. This measures exo-specificity by way of a ratio between the endoamylase activity to the exoamylase activity. Thus, in a preferred aspect, the PS4 variants described here will have less than 0.5 endoamylase units (EAU) per unit of exoamylase activity. Preferably the non-maltogenic exoamylases which are suitable for use according to the present invention have less than 0.05 EAU per unit of exoamylase activity and more preferably less than 0.01 EAU per unit of exoamylase activity.

The PS4 variants described here will preferably have exospecificity, for example measured by exo-specificity indices, as described above, consistent with their being exoamylases. Moreoever, they preferably have higher or increased exospecificity when compared to the parent enzymes or polypeptides from which they are derived. Thus, for example, the PS4 variant polypeptides may have 10%, 20%, 30%, 40%, 50%, 60%, 70%, 80%, 90%, 100%, 200% or higher exo-specificity index when compared to their parent polypeptides, preferably under identical conditions. They may have 1.5× or higher, 2× or higher, 5× or higher, 10× or higher, 50× or higher, 100× or higher, when compared to their parent polypeptides, preferably under identical conditions.

Uses of PS4 Variant Polypeptides and Nucleic Acids

The PS4 variant polypeptides, nucleic acids, host cells, expression vectors, etc, may be used in any application for which an amylase may be used. In particular, they may be used to substitute for any non-maltogenic exoamylase. They may be used to supplement amylase or non-maltogenic exoamylase activity, whether alone or in combination with other known amylases or non-maltogenic exoamylases.

The PS4 variant sequences described here may be used in various applications in the food industry—such as in bakery and drink products, they may also be used in other applications such as a pharmaceutical composition, or even in the chemical industry. In particular, the PS4 variant polypeptides and nucleic acids are useful for various industrial applications including baking (as disclosed in WO 99/50399) and flour standardisation (volume enhancement or improvement). They may be used to produce maltotetraose from starch and other substrates.

We therefore describe a method for preparing a food product, the method comprising: (a) obtaining a non-maltogenic exoamylase; (b) introducing a mutation at a position 161 of the non-maltogenic exoamylase; (c) admixing the resulting polypeptide with a food ingredient.

The PS4 variant polypeptides may be used to enhance the volume of bakery products such as bread. While not wishing to be bound by any particular theory, we believe that this results from the reduction in viscosity of the dough during heating (such as baking) as a result of the exoamylase shortening amylose molecules. This enables the carbon dioxide generated by fermentation to increase the size of the bread with less hindrance.

Thus, food products comprising or treated with PS4 variant polypeptides are expanded in volume when compared to products which have not been so treated, or treated with parent polypeptides. In other words, the food products have a larger volume of air per volume of food product. Alternatively, or in addition, the food products treated with PS4 variant polypeptides have a lower density, or weight (or mass) per volume ratio. In particularly preferred embodiments, the PS4 variant polypeptides are used to enhance the volume of bread. Volume enhancement or expansion is beneficial because it reduces the gumminess or starchiness of foods. Light foods are preferred by consumers, and the customer experience is enhanced. In preferred embodiments, the use of PS4 variant polypeptides enhances the volume by 10%, 20%, 30% 40%, 50% or more.

The use of PS4 variant polypeptides to increase the volume of foods is described in detail in the Examples.

Food Uses

The PS4 variant polypeptides and nucleic acids described here may be used as—or in the preparation of—a food. In particular, they may be added to a food, i.e., as a food additive. The term “food” is intended to include both prepared food, as well as an ingredient for a food, such as a flour. In a preferred aspect, the food is for human consumption. The food may be in the from of a solution or as a solid—depending on the use and/or the mode of application and/or the mode of administration.

The PS4 variant polypeptides and nucleic acids may be used as a food ingredient. As used herein the term “food ingredient” includes a formulation, which is or can be added to functional foods or foodstuffs and includes formulations which can be used at low levels in a wide variety of products that require, for example, acidifying or emulsifying. The food ingredient may be in the from of a solution or as a solid—depending on the use and/or the mode of application and/or the mode of administration.

The PS4 variant polypeptides and nucleic acids disclosed here may be—or may be added to—food supplements. The PS4 variant polypeptides and nucleic acids disclosed here may be—or may be added to—functional foods. As used herein, the term “functional food” means food which is capable of providing not only a nutritional effect and/or a taste satisfaction, but is also capable of delivering a further beneficial effect to consumer. Although there is no legal definition of a functional food, most of the parties with an interest in this area agree that they are foods marketed as having specific health effects.

The PS4 variant polypeptides may also be used in the manufacture of a food product or a foodstuff. Typical foodstuffs include dairy products, meat products, poultry products, fish products and dough products. The dough product may be any processed dough product, including fried, deep fried, roasted, baked, steamed and boiled doughs, such as steamed bread and rice cakes. In highly preferred embodiments, the food product is a bakery product.

Preferably, the foodstuff is a bakery product. Typical bakery (baked) products include bread—such as loaves, rolls, buns, pizza bases etc. pastry, pretzels, tortillas, cakes, cookies, biscuits, krackers etc.

We therefore describe a method of modifying a food additive comprising a non-maltogenic exoamylase, the method comprising introducing a mutation at a position 161 of the non-maltogenic exoamylase. The same method can be used to modify a food ingredient, or a food supplement, a food product, or a foodstuff.

Retrogradation/Staling

We describe the use of PS4 variant proteins that are capable of retarding the staling of starch media, such as starch gels. The PS4 variant polypeptides are especially capable of retarding the detrimental retrogradation of starch.

Most starch granules are composed of a mixture of two polymers: an essentially linear amylose and a highly branched amylopectin. Amylopectin is a very large, branched molecule consisting of chains of α-D-glucopyranosyl units joined by (1-4) linkages, wherein said chains are attached by α-D-(1-6) linkages to form branches. Amylopectin is present in all natural starches, constituting about 75% of most common starches. Amylose is essentially a linear chain of (1-4) linked α-D-glucopyranosyl units having few α-D-(1-6) branches. Most starches contain about 25% amylose.

Starch granules heated in the presence of water undergo an order-disorder phase transition called gelatinization, where liquid is taken up by the swelling granules. Gelatinization temperatures vary for different starches. Upon cooling of freshly baked bread the amylose fraction, within hours, retrogrades to develop a network. This process is beneficial in that it creates a desirable crumb structure with a low degree of firmness and improved slicing properties. More gradually crystallisation of amylopectin takes place within the gelatinised starch granules during the days after baking. In this process amylopectin is believed to reinforce the amylose network in which the starch granules are embedded. This reinforcement leads to increased firmness of the bread crumb. This reinforcement is one of the main causes of bread staling.

It is known that the quality of baked products gradually deteriorates during storage As a consequence of starch recystallisation (also called retrogradation), the water-holding capacity of the crumb is changed with important implications on the organoleptic and dietary properties. The crumb loses softness and elasticity and becomes firm and crumbly. The increase in crumb firmness is often used as a measure of the staling process of bread.

The rate of detrimental retrogradation of amylopectin depends on the length of the side chains of amylopectin. Thus, enzymatic hydrolysis of the amylopectin side chains, for example, by PS4 variant polypeptides having non-maltogenic exoamylase activity, can markedly reduce their crystallisation tendencies.

Accordingly, the use of PS4 variant polypeptides as described here when added to the starch at any stage of its processing into a food product, e.g., before during or after baking into bread can retard or impede or slow down the retrogradation. Such use is described in further detail below.

We therefore describe a method of improving the ability of a non-maltogenic exoamylase to prevent staling, preferably detrimental retrogradation, of a dough product, the method comprising introducing a mutation at a position 161 of the non-maltogenic exoamylase.

Assays for Measurement of Retrogradation (Inc. Staling)

For evaluation of the antistaling effect of the PS4 variant polypeptides having non-maltogenic exoamylase activity described here, the crumb firmness can be measured 1, 3 and 7 days after baking by means of an Instron 4301 Universal Food Texture Analyzer or similar equipment known in the art.

Another method used traditionally in the art and which is used to evaluate the effect on starch retrogradation of a PS4 variant polypeptide having non-maltogenic exoamylase activity is based on DSC (differential scanning calorimetry). Here, the melting enthalpy of retrograded amylopectin in bread crumb or crumb from a model system dough baked with or without enzymes (control) is measured. The DSC equipment applied in the described examples is a Mettler-Toledo DSC 820 run with a temperature gradient of 10° C. per min. from 20 to 95° C. For preparation of the samples 10-20 mg of crumb are weighed and transferred into Mettler-Toledo aluminium pans which then are hermetically sealed.

The model system doughs used in the described examples contain standard wheat flour and optimal amounts of water or buffer with or without the non-maltogenic PS4 variant exoamylase. They are mixed in a 10 or 50 g Brabender Farinograph for 6 or 7 min., respectively. Samples of the doughs are placed in glass test tubes (15*0.8 cm) with a lid. These test tubes are subjected to a baking process in a water bath starting with 30 min. incubation at 33° C. followed by heating from 33 to 95° C. with a gradient of 1.1° C. per min. and finally a 5 min. incubation at 95° C. Subsequently, the tubes are stored in a thermostat at 20° C. prior to DSC analysis.

In preferred embodiments, the PS4 variants described here have a reduced melting enthalpy, compared to the control. In highly preferred embodiments, the PS4 variants have a 10% or more reduced melting enthalpy. Preferably, they have a 20% or more, 30%, 40%, 50%, 60%, 70%, 80%, 90% or more reduced melting enthalpy when compared to the control.

TABLE 2

| | DSC (J/g) |
|---|---|
| Control | 2.29 |
| 0.5 D34 | 1.91 |
| 1 D34 | 1.54 |
| 2 D34 | 1.14 |

The above Table 2 shows DSC values of model dough systems prepared with different doses of PSac-D34 after 7 days of storage. 0.5, 1 and 2 parts per million (or microgram per gram) of flour are tested.

Preparation of Starch Products

We provide the use of PS4 variant polypeptides in the preparation of food products, in particular, starch products. The method comprises forming the starch product by adding a non-maltogenic exoamylase enzyme such as a PS4 variant polypeptide, to a starch medium. If the starch medium is a dough, then the dough is prepared by mixing together flour, water, the non-maltogenic exoamylase which is a PS4 variant polypeptide and optionally other possible ingredients and additives.

The term “starch” should be taken to mean starch per se or a component thereof, especially amylopectin. The term “starch medium” means any suitable medium comprising starch. The term “starch product” means any product that contains or is based on or is derived from starch. Preferably, the starch product contains or is based on or is derived from starch obtained from wheat flour. The term “flour” as used herein is a synonym for the finely-ground meal of wheat or other grain. Preferably, however, the term means flour obtained from wheat per se and not from another grain. Thus, and unless otherwise expressed, references to “wheat flour” as used herein preferably mean references to wheat flour per se as well as to wheat flour when present in a medium, such as a dough.

A preferred flour is wheat flour or rye flour or mixtures of wheat and rye flour. However, dough comprising flour derived from other types of cereals such as for example from rice, maize, barley, and durra are also contemplated. Preferably, the starch product is a bakery product. More preferably, the starch product is a bread product. Even more preferably, the starch product is a baked farinaceous bread product. The term “baked farinaceous bread product” refers to any baked product based on a dough obtainable by mixing flour, water, and a leavening agent under dough forming conditions. Further components can of course be added to the dough mixture.

Thus, if the starch product is a baked farinaceous bread product, then the process comprises mixing—in any suitable order—flour, water, and a leavening agent under dough forming conditions and further adding a PS4 variant polypeptide, optionally in the form of a premix. The leavening agent may be a chemical leavening agent such as sodium bicarbonate or any strain of Saccharomyces cerevisiae (Baker's Yeast).

The PS4 variant non-maltogenic exoamylase can be added together with any dough ingredient including the water or dough ingredient mixture or with any additive or additive mixture. The dough can be prepared by any conventional dough preparation method common in the baking industry or in any other industry making flour dough based products.

Baking of farinaceous bread products such as for example white bread, bread made from bolted rye flour and wheat flour, rolls and the like is typically accomplished by baking the bread dough at oven temperatures in the range of from 180 to 250° C. for about 15 to 60 minutes. During the baking process a steep temperature gradient (200→120° C.) is prevailing in the outer dough layers where the characteristic crust of the baked product is developed. However, owing to heat consumption due to steam generation, the temperature in the crumb is only close to 100° C. at the end of the baking process.

We therefore describe a process for making a bread product comprising: (a) providing a starch medium; (b) adding to the starch medium a PS4 variant polypeptide as described in this document; and (c) applying heat to the starch medium during or after step (b) to produce a bread product. We also describe a process for making a bread product comprising adding to a starch medium a PS4 variant polypeptide as described.

The non-maltogenic exoamylase PS4 variant polypeptide can be added as a liquid preparation or as a dry pulverulent composition either comprising the enzyme as the sole active component or in admixture with one or more additional dough ingredient or dough additive.

Improving Composition

We describe improver compositions, which include bread improving compositions and dough improving compositions. These comprise a PS4 variant polypeptide, optionally together with a further ingredient, or a further enzyme, or both.

We also provide for the use of such a bread and dough improving compositions in baking. In a further aspect, we provide a baked product or dough obtained from the bread improving composition or dough improving composition. In another aspect, we describe a baked product or dough obtained from the use of a bread improving composition or a dough improving composition.

Dough Preparation

A dough may be prepared by admixing flour, water, a dough improving composition comprising PS4 variant polypeptide (as described above) and optionally other ingredients and additives.

The dough improving composition can be added together with any dough ingredient including the flour, water or optional other ingredients or additives. The dough improving composition can be added before the flour or water or optional other ingredients and additives. The dough improving composition can be added after the flour or water, or optional other ingredients and additives. The dough can be prepared by any conventional dough preparation method common in the baking industry or in any other industry making flour dough based products.

The dough improving composition can be added as a liquid preparation or in the form of a dry powder composition either comprising the composition as the sole active component or in admixture with one or more other dough ingredients or additive.

The amount of the PS4 variant polypeptide non-maltogenic exoamylase that is added is normally in an amount which results in the presence in the finished dough of 50 to 100,000 units per kg of flour, preferably 100 to 50,000 units per kg of flour. Preferably, the amount is in the range of 200 to 20,000 units per kg of flour.

In the present context, 1 unit of the non-maltogenic exoamylase is defined as the amount of enzyme which releases hydrolysis products equivalent to 1 μmol of reducing sugar per min. when incubated at 50 degrees C. in a test tube with 4 ml of 10 mg/ml waxy maize starch in 50 mM MES, 2 mM calcium chloride, pH 6.0 as described hereinafter.

The dough as described here generally comprises wheat meal or wheat flour and/or other types of meal, flour or starch such as corn flour, corn starch, maize flour, rice flour, rye meal, rye flour, oat flour, oat meal, soy flour, sorghum meal, sorghum flour, potato meal, potato flour or potato starch. The dough may be fresh, frozen, or part-baked.

The dough may be a leavened dough or a dough to be subjected to leavening. The dough may be leavened in various ways, such as by adding chemical leavening agents, e.g., sodium bicarbonate or by adding a leaven (fermenting dough), but it is preferred to leaven the dough by adding a suitable yeast culture, such as a culture of Saccharomyces cerevisiae (baker's yeast), e.g. a commercially available strain of S. cerevisiae.

The dough may comprise fat such as granulated fat or shortening. The dough may further comprise a further emulsifier such as mono- or diglycerides, sugar esters of fatty acids, polyglycerol esters of fatty acids, lactic acid esters of monoglycerides, acetic acid esters of monoglycerides, polyoxethylene stearates, or lysolecithin.

We also describe a pre-mix comprising flour together with the combination as described herein. The pre-mix may contain other dough-improving and/or bread-improving additives, e.g. any of the additives, including enzymes, mentioned herein.

Further Dough Additives or Ingredients

In order to improve further the properties of the baked product and impart distinctive qualities to the baked product further dough ingredients and/or dough additives may be incorporated into the dough. Typically, such further added components may include dough ingredients such as salt, grains, fats and oils, sugar or sweeteber, dietary fibres, protein sources such as milk powder, gluten soy or eggs and dough additives such as emulsifiers, other enzymes, hydrocolloids, flavouring agents, oxidising agents, minerals and vitamins

The emulsifiers are useful as dough strengtheners and crumb softeners. As dough strengtheners, the emulsifiers can provide tolerance with regard to resting time and tolerance to shock during the proofing. Furthermore, dough strengtheners will improve the tolerance of a given dough to variations in the fermentation time. Most dough strengtheners also improve on the oven spring which means the increase in volume from the proofed to the baked goods. Lastly, dough strengtheners will emulsify any fats present in the recipe mixture.

Suitable emulsifiers include lecithin, polyoxyethylene stearat, mono- and diglycerides of edible fatty acids, acetic acid esters of mono- and diglycerides of edible fatty acids, lactic acid esters of mono- and diglycerides of edible fatty acids, citric acid esters of mono- and diglycerides of edible fatty acids, diacetyl tartaric acid esters of mono- and diglycerides of edible fatty acids, sucrose esters of edible fatty acids, sodium stearoyl-2-lactylate, and calcium stearoyl-2-lactylate.

The further dough additive or ingredient can be added together with any dough ingredient including the flour, water or optional other ingredients or additives, or the dough improving composition. The further dough additive or ingredient can be added before the flour, water, optional other ingredients and additives or the dough improving composition. The further dough additive or ingredient can be added after the flour, water, optional other ingredients and additives or the dough improving composition.

The further dough additive or ingredient may conveniently be a liquid preparation. However, the further dough additive or ingredient may be conveniently in the form of a dry composition.

Preferably the further dough additive or ingredient is at least 1% the weight of the flour component of dough. More preferably, the further dough additive or ingredient is at least 2%, preferably at least 3%, preferably at least 4%, preferably at least 5%, preferably at least 6%. If the additive is a fat, then typically the fat may be present in an amount of from 1 to 5%, typically 1 to 3%, more typically about 2%.

Further Enzyme

In addition to the PS4 variant polypeptides, one or more further enzymes may be used, for example added to the food, dough preparation, foodstuff or starch composition.

Further enzymes that may be added to the dough include oxidoreductases, hydrolases, such as lipases and esterases as well as glycosidases like α-amylase, pullulanase, and xylanase. Oxidoreductases, such as for example glucose oxidase and hexose oxidase, can be used for dough strengthening and control of volume of the baked products and xylanases and other hemicellulases may be added to improve dough handling properties, crumb softness and bread volume. Lipases are useful as dough strengtheners and crumb softeners and α-amylases and other amylolytic enzymes may be incorporated into the dough to control bread volume and further reduce crumb firmness.

Further enzymes that may be used may be selected from the group consisting of a cellulase, a hemicellulase, a starch degrading enzyme, a protease, a lipoxygenase.

Examples of useful oxidoreductases include oxidises sush as maltose oxidising enzyme, a glucose oxidase (EC 1.1.3.4), carbohydrate oxidase, glycerol oxidase, pyranose oxidase, galactose oxidase (EC 1.1.3.10) and hexose oxidase (EC 1.1.3.5).

Among starch degrading enzymes, amylases are particularly useful as dough improving additives. α-amylase breaks downs starch into dextrins which are further broken down by β-amylase to maltose. Other useful starch degrading enzymes which may be added to a dough composition include glucoamylases and pullulanases.

Preferably, the further enzyme is at least a xylanase and/or at least an amylase. The term “xylanase” as used herein refers to xylanases (EC 3.2.1.32) which hydrolyse xylosidic linkages.

The term “amylase” as used herein refers to amylases such as α-amylases (EC 3.2.1.1), β-amylases (EC 3.2.1.2) and γ-amylases (EC 3.2.1.3.

The further enzyme can be added together with any dough ingredient including the flour, water or optional other ingredients or additives, or the dough improving composition. The further enzyme can be added before the flour, water, and optionally other ingredients and additives or the dough improving composition. The further enzyme can be added after the flour, water, and optionally other ingredients and additives or the dough improving composition. The further enzyme may conveniently be a liquid preparation. However, the composition may be conveniently in the form of a dry composition.

Some enzymes of the dough improving composition are capable of interacting with each other under the dough conditions to an extent where the effect on improvement of the rheological and/or machineability properties of a flour dough and/or the quality of the product made from dough by the enzymes is not only additive, but the effect is synergistic.

In relation to improvement of the product made from dough (finished product), it may be found that the combination results in a substantial synergistic effect in respect to crumb structore. Also, with respect to the specific volume of baked product a synergistic effect may be found.

The further enzyme may be a lipase (EC 3.1.1) capable of hydrolysing carboxylic ester bonds to release carboxylate. Examples of lipases include but are not limited to triacylglycerol lipase (EC 3.1.1.3), galactolipase (EC 3.1.1.26), phospholipase A1 (EC 3.1.1.32, phospholipase A2 (EC 3.1.1.4) and lipoprotein lipase A2 (EC 3.1.1.34).

Amylase Combinations

We disclose in particular combinations of PS4 variant polypeptides with amylases, in particular, maltogenic amylases. Maltogenic alpha-amylase (glucan 1,4-a-maltohydrolase, E.C. 3.2.1.133) is able to hydrolyze amylose and amylopectin to maltose in the alpha-configuration.

A maltogenic alpha-amylase from Bacillus (EP 120 693) is commercially available under the trade name Novamyl (Novo Nordisk A/S, Denmark) and is widely used in the baking industry as an anti-staling agent due to its ability to reduce retrogradation of starch. Novamyl is described in detail in International Patent Publication WO 91/04669. The maltogenic alpha-amylase Novamyl shares several characteristics with cyclodextrin glucanotransferases (CGTases), including sequence homology (Henrissat B, Bairoch A; Biochem. J., 316, 695-696 (1996)) and formation of transglycosylation products (Christophersen, C., et al., 1997, Starch, vol. 50, No. 1, 39-45).

In highly preferred embodiments, we disclose combinations comprising PS4 variant polypeptides together with Novamyl or any of its variants. Such combinations are useful for food production such as baking. The Novamyl may in particular comprise Novamyl 1500 MG.

Other documents describing Novamyl and its uses include Christophersen, C., Pedersen, S., and Christensen, T., (1993) Method for production of maltose an a limit dextrin, the limit dextrin, and use of the limit dextrin. Denmark, and WO 95/10627. It is further described in U.S. Pat. No. 4,598,048 and U.S. Pat. No. 4,604,355. Each of these documents is hereby incorporated by reference, and any of the Novamyl polypeptides described therein may be used in combinations with any of the PS4 variant polypeptides described here.

Variants, homologues, and mutants of Novamyl may be used for the combinations, provided they retain alpha amylase activity. For example, any of the Novamyl variants disclosed in U.S. Pat. No. 6,162,628, the entire disclosure of which is hereby incorporated by reference, may be used in combination with the PS4 variant polypeptides described here. In particular, any of the polypeptides described in that document, specifically variants of SEQ ID NO:1 of U.S. Pat. No. 6,162,628 at any one or more positions corresponding to Q13, I16, D17, N26, N28, P29, A30, S32, Y33, G34, L35, K40, M45, P73, V74, D76 N77, D79, N86, R95, N99, I100, H103, Q119, N120, N131, S141, T142, A148, N152, A163, H169, N171, G172, I174, N176, N187, F188, A192, Q201, N203, H220, N234, G236, Q247, K249, D261, N266, L268, R272, N275, N276, V279, N280, V281, D285, N287, F297, Q299, N305, K316, N320, L321, N327, A341, N342, A348, Q365, N371, N375, M378, G397, A381, F389, N401, A403, K425, N436, S442, N454, N468, N474, S479, A483, A486, V487, S493, T494, S495, A496, S497, A498, Q500, N507, 1510, N513, K520, Q526, A555, A564, S573, N575, Q581, S583, F586, K589, N595, G618, N621, Q624, A629, F636, K645, N664 and/or T681 may be used.

Amino Acid Sequences

The invention makes use of a PS4 variant nucleic acid, and the amino acid sequences of such PS4 variant nucleic acids are encompassed by the methods and compositions described here.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The amino acid sequence may be prepared/isolated from a suitable source, or it may be made synthetically or it may be prepared by use of recombinant DNA techniques.

The PS4 variant enzyme described here may be used in conjunction with other enzymes. Thus we further disclose a combination of enzymes wherein the combination comprises a PS4 variant polypeptide enzyme described here and another enzyme, which itself may be another PS4 variant polypeptide enzyme.

PS4 Variant Nucleotide Sequence

As noted above, we disclose nucleotide sequences encoding the PS4 variant enzymes having the specific properties described.

The term “nucleotide sequence” or “nucleic acid sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or anti-sense strand.

The term “nucleotide sequence” as used in this document includes genomic DNA, cDNA, synthetic DNA, and RNA. Preferably it means DNA, more preferably cDNA sequence coding for a PS4 variant polypeptide.

Typically, the PS4 variant nucleotide sequence is prepared using recombinant DNA techniques (i.e. recombinant DNA). However, in an alternative embodiment, the nucleotide sequence could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232).

Preparation of Nucleic Acid Sequences

A nucleotide sequence encoding either an enzyme which has the specific properties as defined herein (e.g., a PS4 variant polypeptide) or an enzyme which is suitable for modification, such as a parent enzyme, may be identified and/or isolated and/or purified from any cell or organism producing said enzyme. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme or a part of the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used.

Alternatively, enzyme-encoding clones could be identified by inserting fragments of genomic DNA into an expression vector, such as a plasmid, transforming enzyme-negative bacteria with the resulting genomic DNA library, and then plating the transformed bacteria onto agar plates containing a substrate for enzyme (i.e. maltose), thereby allowing clones expressing the enzyme to be identified.

In a yet further alternative, the nucleotide sequence encoding the enzyme may be prepared synthetically by established standard methods, e.g. the phosphoroamidite method described by Beucage S. L. et al., (1981) Tetrahedron Letters 22, p 1859-1869, or the method described by Matthes et al., (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487-491).

Variants/Homologues/Derivatives

We further describe the use of variants, homologues and derivatives of any amino acid sequence of an enzyme or of any nucleotide sequence encoding such an enzyme, such as a PS4 variant polypeptide or a PS4 variant nucleic acid. Unless the context dictates otherwise, the term “PS4 variant nucleic acid” should be taken to include each of the nucleic acid entities described below, and the term “PS4 variant polypeptide” should likewise be taken to include each of the polypeptide or amino acid entities described below.

Here, the term “homologue” means an entity having a certain homology with the subject amino acid sequences and the subject nucleotide sequences. Here, the term “homology” can be equated with “identity”.

In the present context, a homologous sequence is taken to include an amino acid sequence which may be at least 75, 80, 85 or 90% identical, preferably at least 95, 96, 97, 98 or 99% identical to the subject sequence. Typically, the homologues will comprise the same active sites etc. as the subject amino acid sequence. Although homology can also be considered in terms of similarity (i.e. amino acid residues having similar chemical properties/functions), in the context of this document it is preferred to express homology in terms of sequence identity.

In the present context, an homologous sequence is taken to include a nucleotide sequence which may be at least 75, 80, 85 or 90% identical, preferably at least 95, 96, 97, 98 or 99% identical to a nucleotide sequence encoding a PS4 variant polypeptide enzyme (such as a PS4 variant nucleic acid). Typically, the homologues will comprise the same sequences that code for the active sites etc as the subject sequence. Although homology can also be considered in terms of similarity (i.e. amino acid residues having similar chemical properties/functions), in the context of this document it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (Devereux et al 1984 Nuc. Acids Research 12 p 387). Examples of other software than can perform sequence comparisons include, but are not limited to, the BLAST package (see Ausubel et al., 1999 Short Protocols in Molecular Biology, 4^{th} Ed—Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60).

However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244).

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids can be grouped together based on the properties of their side chain alone. However it is more useful to include mutation data as well. The sets of amino acids thus derived are likely to be conserved for structural reasons. These sets can be described in the form of a Venn diagram (Livingstone C. D. and Barton G. J. (1993) “Protein sequence alignments: a strategy for the hierarchical analysis of residue conservation” Comput. Appl Biosci. 9: 745-756) (Taylor W. R. (1986) “The classification of amino acid conservation” J. Theor. Biol. 119; 205-218). Conservative substitutions may be made, for example according to the table below which describes a generally accepted Venn diagram grouping of amino acids.

| Set | | Sub-set | |
|---|---|---|---|
| Hydrophobic | F W Y H K M I L V A G C | Aromatic | F W Y H |
| | | Aliphatic | I L V |
| Polar | W Y H K R E D C S T N Q | Charged | H K R E D |
| | | Positively | H K R |
| | | charged | |
| | | Negatively | E D |
| | | charged | |
| Small | V C A G S P T N D | Tiny | A G S |

We further disclose sequences comprising homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as 0), pyriylalanine, thienylalanine, naphthylalanine and phenylglycine.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleotide sequences described here, and suitable for use in the methods and compositions described here (such as PS4 variant nucleic acids) may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of this document, it is to be understood that the nucleotide sequences described herein may be modified by any method available in the art. Such modifications may be carried out in order to enhance the in vivo activity or life span of nucleotide sequences.

We further describe the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the PS4 variant sequences may be obtained in a number of ways. Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other species, and probing such libraries with probes comprising all or part of any one of the sequences in the attached sequence listings under conditions of medium to high stringency. Similar considerations apply to obtaining species homologues and allelic variants of the polypeptide or nucleotide sequences described here.

Variants and strain/species homologues may also be obtained using degenerate PCR which will use primers designed to target sequences within the variants and homologues encoding conserved amino acid sequences. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The polynucleotides (nucleotide sequences) such as the PS4 variant nucleic acids described in this document may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides.

Polynucleotides such as DNA polynucleotides and probes may be produced recombinantly, synthetically, or by any means available to those of skill in the art. They may also be cloned by standard techniques. In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction enzyme recognition sites so that the amplified DNA can be cloned into a suitable cloning vector. Preferably, the variant sequences etc. are at least as biologically active as the sequences presented herein.

As used herein “biologically active” refers to a sequence having a similar structural function (but not necessarily to the same degree), and/or similar regulatory function (but not necessarily to the same degree), and/or similar biochemical function (but not necessarily to the same degree) of the naturally occurring sequence.

Hybridisation

We further describe sequences that are complementary to the nucleic acid sequences of PS4 variants or sequences that are capable of hybridising either to the PS4 variant sequences or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies. Therefore, we disclose the use of nucleotide sequences that are capable of hybridising to the sequences that are complementary to the sequences presented herein, or any derivative, fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under stringent conditions (e.g. 50° C. and 0.2×SSC {1×SSC=0.15 M NaCl, 0.015 M Na₃citrate pH 7.0}) to the nucleotide sequences presented herein. More preferably, the term “variant” encompasses sequences that are complementary to sequences that are capable of hybridising under high stringent conditions (e.g. 65° C. and 0.1×SSC {1×SSC=0.15 M NaCl, 0.015 M Na₃citrate pH 7.0}) to the nucleotide sequences presented herein.

We further disclose nucleotide sequences that can hybridise to the nucleotide sequences of PS4 variants (including complementary sequences of those presented herein), as well as nucleotide sequences that are complementary to sequences that can hybridise to the nucleotide sequences of PS4 variants (including complementary sequences of those presented herein). We further describe polynucleotide sequences that are capable of hybridising to the nucleotide sequences presented herein under conditions of intermediate to maximal stringency.

In a preferred aspect, we disclose nucleotide sequences that can hybridise to the nucleotide sequence of a PS4 variant nucleic acid, or the complement thereof, under stringent conditions (e.g. 50° C. and 0.2×SSC). More preferably, the nucleotide sequences can hybridise to the nucleotide sequence of a PS4 variant, or the complement thereof, under high stringent conditions (e.g. 65° C. and 0.1×SSC).

Site-Directed Mutagenesis

Once an enzyme-encoding nucleotide sequence has been isolated, or a putative enzyme-encoding nucleotide sequence has been identified, it may be desirable to mutate the sequence in order to prepare an enzyme. Accordingly, a PS4 variant sequence may be prepared from a parent sequence. Mutations may be introduced using synthetic oligonucleotides. These oligonucleotides contain nucleotide sequences flanking the desired mutation sites.

A suitable method is disclosed in Morinaga et al., (Biotechnology (1984) 2, p 646-649). Another method of introducing mutations into enzyme-encoding nucleotide sequences is described in Nelson and Long (Analytical Biochemistry (1989), 180, p 147-151). A further method is described in Sarkar and Sommer (Biotechniques (1990), 8, p 404-407—“The megaprimer method of site directed mutagenesis”).

In one aspect the sequence for use in the methods and compositions described here is a recombinant sequence—i.e. a sequence that has been prepared using recombinant DNA techniques. These recombinant DNA techniques are within the capabilities of a person of ordinary skill in the art. Such techniques are explained in the literature, for example, J. Sambrook, E. F. Fritsch, and T. Maniatis, 1989, Molecular Cloning: A Laboratory Manual, Second Edition, Books 1-3, Cold Spring Harbor Laboratory Press.

In one aspect the sequence for use in the methods and compositions described here is a synthetic sequence—i.e. a sequence that has been prepared by in vitro chemical or enzymatic synthesis. It includes, but is not limited to, sequences made with optimal codon usage for host organisms—such as the methylotrophic yeasts Pichia and Hansenula.

The nucleotide sequence for use in the methods and compositions described here may be incorporated into a recombinant replicable vector. The vector may be used to replicate and express the nucleotide sequence, in enzyme form, in and/or from a compatible host cell. Expression may be controlled using control sequences eg. regulatory sequences. The enzyme produced by a host recombinant cell by expression of the nucleotide sequence may be secreted or may be contained intracellularly depending on the sequence and/or the vector used. The coding sequences may be designed with signal sequences which direct secretion of the substance coding sequences through a particular prokaryotic or eukaryotic cell membrane.

Expression of PS4 Nucleic Acids and Polypeptides

The PS4 polynucleotides and nucleic acids may include DNA and RNA of both synthetic and natural origin which DNA or RNA may contain modified or unmodified deoxy- or dideoxy-nucleotides or ribonucleotides or analogs thereof. The PS4 nucleic acid may exist as single- or double-stranded DNA or RNA, an RNA/DNA heteroduplex or an RNA/DNA copolymer, wherein the term “copolymer” refers to a single nucleic acid strand that comprises both ribonucleotides and deoxyribonucleotides. The PS4 nucleic acid may even be codon optimised to further increase expression.

The term “synthetic”, as used herein, is defined as that which is produced by in vitro chemical or enzymatic synthesis. It includes but is not limited to PS4 nucleic acids made with optimal codon usage for host organisms such as the the methylotrophic yeasts Pichia and Hansenula.

Polynucleotides, for example variant PS4 polynucleotides described here, can be incorporated into a recombinant replicable vector. The vector may be used to replicate the nucleic acid in a compatible host cell. The vector comprising the polynucleotide sequence may be transformed into a suitable host cell. Suitable hosts may include bacterial, yeast, insect and fungal cells.

The term “transformed cell” includes cells that have been transformed by use of recombinant DNA techniques. The transformation typically occurs by insertion of one or more nucleotide sequences into a cell that is to be transformed. The inserted nucleotide sequence may be a heterologous nucleotide sequence (i.e. is a sequence that is not natural to the cell that is to be transformed. In addition, or in the alternative, the inserted nucleotide sequence may be an homologous nucleotide sequence (i.e. is a sequence that is natural to the cell that is to be transformed)—so that the cell receives one or more extra copies of a nucleotide sequence already present in it.

Thus in a further embodiment, we provide a method of making PS4 variant polypeptides and polynucleotides by introducing a polynucleotide into a replicable vector, introducing the vector into a compatible host cell, and growing the host cell under conditions which bring about replication of the vector. The vector may be recovered from the host cell.

Expression Constructs

The PS4 nucleic acid may be operatively linked to transcriptional and translational regulatory elements active in a host cell of interest. The PS4 nucleic acid may also encode a fusion protein comprising signal sequences such as, for example, those derived from the glucoamylase gene from Schwanniomyces occidentalis, α-factor mating type gene from Saccharomyces cerevisiae and the TAKA-amylase from Aspergillus oryzae. Alternatively, the PS4 nucleic acid may encode a fusion protein comprising a membrane binding domain.

Expression Vector

The PS4 nucleic acid may be expressed at the desired levels in a host organism using an expression vector.

An expression vector comprising a PS4 nucleic acid can be any vector which is capable of expressing the gene encoding PS4 nucleic acid in the selected host organism, and the choice of vector will depend on the host cell into which it is to be introduced. Thus, the vector can be an autonomously replicating vector, i.e. a vector that exists as an episomal entity, the replication of which is independent of chromosomal replication, such as, for example, a plasmid, a bacteriophage or an episomal element, a minichromosome or an artificial chromosome. Alternatively, the vector may be one which, when introduced into a host cell, is integrated into the host cell genome and replicated together with the chromosome.

Components of the Expression Vector

The expression vector typically includes the components of a cloning vector, such as, for example, an element that permits autonomous replication of the vector in the selected host organism and one or more phenotypically detectable markers for selection purposes. The expression vector normally comprises control nucleotide sequences encoding a promoter, operator, ribosome binding site, translation initiation signal and optionally, a repressor gene or one or more activator genes. Additionally, the expression vector may comprise a sequence coding for an amino acid sequence capable of targeting the PS4 variant polypeptide to a host cell organelle such as a peroxisome or to a particular host cell compartment. Such a targeting sequence includes but is not limited to the sequence SKL. In the present context, the term “expression signal” includes any of the above control sequences, repressor or activator sequences. For expression under the direction of control sequences, the nucleic acid sequence the PS4 variant polypeptide is operably linked to the control sequences in proper manner with respect to expression.

Preferably, a polynucleotide in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators. The control sequences may in particular comprise promoters.

Promoter

In the vector, the nucleic acid sequence encoding for the variant PS4 polypeptide is operably combined with a suitable promoter sequence. The promoter can be any DNA sequence having transcription activity in the host organism of choice and can be derived from genes that are homologous or heterologous to the host organism.

Bacterial Promoters

Examples of suitable promoters for directing the transcription of the modified nucleotide sequence, such as PS4 nucleic acids, in a bacterial host include the promoter of the lac operon of E. coli, the Streptomyces coelicolor agarase gene dagA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase gene (amyQ), the promoters of the Bacillus subtilis xylA and xylB genes and a promoter derived from a Lactococcus sp.-derived promoter including the P170 promoter. When the gene encoding the PS4 variant polypeptide is expressed in a bacterial species such as E. coli, a suitable promoter can be selected, for example, from a bacteriophage promoter including a T7 promoter and a phage lambda promoter.

Fungal Promoters

For transcription in a fungal species, examples of useful promoters are those derived from the genes encoding the, Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, Aspergillus oryzae alkaline protease, Aspergillus oryzae triose phosphate isomerase or Aspergillus nidulans acetamidase.

Yeast Promoters

Examples of suitable promoters for the expression in a yeast species include but are not limited to the Gal 1 and Gal 10 promoters of Saccharomyces cerevisiae and the Pichia pastoris AOX1 or AOX2 promoters.

Host Organisms

(I) Bacterial Host Organisms

Examples of suitable bacterial host organisms are gram positive bacterial species such as Bacillaceae including Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus lautus, Bacillus megaterium and Bacillus thuringiensis, Streptomyces species such as Streptomyces murinus, lactic acid bacterial species including Lactococcus spp. such as Lactococcus lactis, Lactobacillus spp. including Lactobacillus reuteri, Leuconostoc spp., Pediococcus spp. and Streptococcus spp. Alternatively, strains of a gram-negative bacterial species belonging to Enterobacteriaceae including E. coli, or to Pseudomonadaceae can be selected as the host organism.

(II) Yeast Host Organisms

A suitable yeast host organism can be selected from the biotechnologically relevant yeasts species such as but not limited to yeast species such as Pichia sp., Hansenula sp or Kluyveromyces, Yarrowinia species or a species of Saccharomyces including Saccharomyces cerevisiae or a species belonging to Schizosaccharomyce such as, for example, S. Pombe species.

Preferably a strain of the methylotrophic yeast species Pichia pastoris is used as the host organism. Preferably the host organism is a Hansenula species.

(III) Fungal Host Organisms

Suitable host organisms among filamentous fungi include species of Aspergillus, e.g. Aspergillus niger, Aspergillus oryzae, Aspergillus tubigensis, Aspergillus awamori or Aspergillus nidulans. Alternatively, strains of a Fusarium species, e.g. Fusarium oxysporum or of a Rhizomucor species such as Rhizomucor miehei can be used as the host organism. Other suitable strains include Thermomyces and Mucor species.

Protein Expression and Purification

Host cells comprising polynucleotides may be used to express polypeptides, such as variant PS4 polypeptides, fragments, homologues, variants or derivatives thereof. Host cells may be cultured under suitable conditions which allow expression of the proteins. Expression of the polypeptides may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Polypeptides can be extracted from host cells by a variety of techniques known in the art, including enzymatic, chemical and/or osmotic lysis and physical disruption. Polypeptides may also be produced recombinantly in an in vitro cell-free system, such as the TnT™ (Promega) rabbit reticulocyte system.

### EXAMPLES

### Example 1

### Cloning of PS4

Pseudomonas sacharophila is grown overnight on LB media and chromosomal DNA is isolated by standard methods (Sambrook J, 1989). A 2190 bp fragment containing the PS4 open reading frame (Zhou et al., 1989) is amplified from P. sacharophila chromosomal DNA by PCR using the primers P1 and P2 (see Table 3). The resulting fragment is used as a template in a nested PCR with primers P3 and P4, amplifying the open reading frame of PS4 without its signal sequence and introducing a NcoI site at the 5′ end of the gene and a BamHI site at the 3′end. Together with the NcoI site a codon for a N-terminal Methionine is introduced, allowing for intracellular expression of PS4. The 1605 bp fragment is cloned into pCRBLUNT TOPO (Invitrogen) and the integrity of the construct analysed by sequencing. The E. coli Bacillus shuttle vector pDP66K (Penninga et al., 1996) is modified to allow for expression of the PS4 under control of the P32 promoter and the ctgase signal sequence. The resulting plasmid, pCSmta is transformed into B. subtilis.

A second expression construct is made in which the starch binding domain of PS4 is removed. In a PCR with primers P3 and P6 (Table 3) on pCSmta, a truncated version of the mta gene is generated. The full length mta gene in pCSmta is exchanged with the truncated version which resulted in the plasmid pCSmta-SBD.

### Example 2

### Site Directed Mutagenesis of PS4

Mutations are introduced into the mta gene by 2 methods. Either by a 2 step PCR based method, or by a Quick Exchange method (QE). For convenience the mta gene is split up in 3 parts; a PvuI-FspI fragment, a FspI-PstI fragment and a PstI-AspI fragment, further on referred to as fragment 1, 2 and 3 respectively.

In the 2 step PCR based method, mutations are introduced using Pfu DNA polymerase (Stratagene). A first PCR is carried out with a mutagenesis primer (Table 4) for the coding strand plus a primer downstream on the lower strand (either 2R or 3R Table 3). The reaction product is used as a primer in a second PCR together with a primer upstream on the coding strand. The product of the last reaction is cloned into pCRBLUNT topo (Invitrogen) and after sequencing the fragment is exchanged with the corresponding fragment in pCSmta.

Using the Quick Exchange method (Stratagene), mutations are introduced using two complementary primers in a PCR on a plasmid containing the mta gene, or part of the mta gene.

For this purpose a convenient set of plasmids is constructed, comprising of 3 SDM plasmids and 3 pCSΔ plasmids. The SDM plasmids each bear 1 of the fragments of the mta gene as mentioned above, in which the desired mutation is introduced by QE. After verification by sequencing, the fragments are cloned into the corresponding recipient pCSΔ plasmid. The pCSΔ plasmids are inactive derivatives from pCSmta. Activity is restored by cloning the corresponding fragment from the SDM plasmid, enabling easy screening.

TABLE 3

Primers used in cloning the mta gene, and
standard primers used in construction of site
directed mutants with the 2 step PCR method.

| | | Introduced |
|---|---|---|
| Primer | Primer sequence | site |
| P1 | 5′- ATG ACG AGG TCC TTG TTT TTC | |
| I. P2 | 5′- CGC TAG TCG TCC ATG TCG | |
| P3 | 5′- GCC ATG GAT CAG GCC GGC AAG AGC CCG | NcoI |
| P4 | 5′- TGG ATC CTC AGA ACG AGC CGC TGG T | BamHI |
| P6 | 5′- GAA TTC AGC CGC CGT CAT TCC CGC C | EcoRI |
| 2L | 5′- AGA TTT ACG GCA TGT TTC GC | |
| 2R | 5′- TAG CCG CTA TGG AAG CTG AT | |
| 3L | 5′- TGA CCT TCG TCG ACA ACC AC | |
| 3R | 5′- GAT AGC TGC TGG TGA CGG TC | |

TABLE 4
Primers used to introduce site directed mutations in mta

| | | | | |
|---|---|---|---|---|
| MUTATION | Oligo Sequence | MODIFICATION | Strand | PURPOSE |

| | | | |
|---|---|---|---|
| G134R | CTGCCGGCCGGCCAGcGCTTCTGGCG | + | SDM |
| G134R- | cgccagaagcgctggccggccggcag | − | SDM |
| I157L | GACGGTGACCGCTTCcTgGGCGGCGAGTCG | + | SDM |
| I151L- | cgactcgccgcccaggaagcggtcaccgtc | − | SDM |
| G223A | GGCGAGCTGTGGAAAgccCCTTCTGAATATCCG | + | SDM |
| G223A- | cggatattcagaaggggctttccacagctcgcc | − | SDM |
| H307L | gaacGGCGGCCAGCACctgTGGGCGCTGCAG | + | SDM |
| H307L- | ctgcagcgcccacaggtgctggccgccgttc | − | SDM |
| S334P, | GTACTGGccgCACATGTACGACTGGGGCTACGGC | + | SDM |
| D343E | gaaTTCATC | | |
| S334P, | gatgaattcgccgtagccccagtcgtacatgtgc | − | SDM |
| D343E- | ggccagtac | | |

TABLE 5
Features of the SDM and pCSΔ plasmids

Plasmid

| | |
|---|---|
| SDM1 | pBlueSK+480 bp SalI-StuI fragment mta |
| SDM2 | pBlueSK+572 bp SacII-PstI fragment mta |
| SDM2 | pBlueSK+471 bp SalI-StuI fragment mta |
| pCSΔ1 | FseI site filled in with Klenow ----> frameshift in mta |
| pCSΔ2 | FspI-PstI fragment of mta replaced with ‘junk-DNA’ |
| pCSΔ3 | PstI-AspI fragment of mta replaced with ‘junk-DNA’ |

### Example 3

### Multi SDM

The PS4 variants were generated using a QuickChange® Multi Site Directed Mutagenesis Kit (Stratagene) according to the manufactures protocol with some modifications as described.

Step 1: Mutant Strand Synthesis Reaction (PCR)

Inoculate 3 ml. LB (22 g/l Lennox L Broth Base, Sigma)+antibiotics (0.05 μg/ml kanamycin, Sigma) in a 10 ml Falcon tube
Incubate o/n 37° C., ca. 200 rpm.
Spin down the cells by centrifugation (5000 rpm/5 min)
Poor off the medium
Prepare ds-DNA template using QIAGEN Plasmid Mini Purification Protocol

1. The mutant strand synthesis reaction for thermal cycling was prepared as follow:

PCR Mix:
2.5 μl 10× QuickChange® Multi reaction buffer
0.75 μl QuickSolution

$$X μl \qquad Primers \begin{matrix}( \begin{matrix}primer length 28 - 35 bp -> 10 pmol \\ primer length 24 - 27 bp -> 7 pmol\end{matrix} ) \\ primer length 20 - 23 bp -> 5 pmol\end{matrix}$$
1 μL dNTP mix
X μl ds-DNA template (200 ng)
1 μl QuickChange® Multi enzyme blend (2.5 U/μl) (PfuTurbo® DNA polymerase)
X μl dH₂O (to a final volume of 25 μl)

Mix all components by pipetting and briefly spin down the reaction mixtures.

2. Cycle the reactions using the following parameters:
35 cycles of denaturation (96° C./1 min)
   primer annealing (62.8° C./1 min)
   elongation (65° C./15 min)
   then hold at 4° C.

Preheat the lid of the PCR machine to 105° C. and the plate to 95° C. before the PCR tubes are placed in the machine (eppendorf thermal cycler).

Step 2: Dpn I Digestion

1. Add 2 μl Dpn I restriction enzyme (10 U/μl) to each amplification reaction, mix by pipetting and spin down mixture.

2. Incubate at 37° C. for ˜3 hr.

Step 3: Transformation of XL10-Gold® Ultracompetent Cells

1. Thaw XL10-Gold cells on ice. Aliquot 45 μl cells per mutagenesis reaction to prechilled Falcon tubes.

2. Turn on the waterbath (42° C.) and place a tube with NZY⁺ broth in the bath to preheat.

3. Add 2 μl β-mercaptoethanol mix to each tube. Swirl and tap gently and incubate 10 min on ice, swirling every 2 min.

4. Add 1.5 μl Dpn I-treated DNA to each aliquot of cells, swirl to mix and incubate on ice for 30 min.

5. Heat-pulse the tubes in 42° C. waterbath for 30 s and place on ice for 2 min.

6. Add 0.5 ml preheated NZY⁺ broth to each tube and incubate at 37° C. for 1 hr with shaking at 225-250 rpm.

7. Plate 200 μl of each transformation reaction on LB plates (33.6 g/l Lennox L Agar, Sigma) containing 1% starch and 0.05 μg/ml kanamycin

8. Incubate the transformation plates at 37° C. overnight.

TABLE 6
Primer table for pPD77d14:

| | | | | |
|---|---|---|---|---|
| MUTATION | Oligo Sequence | MODIFICATION | Strand | PURPOSE |

| | | | | |
|---|---|---|---|---|
| N33Y, | GCGAAGCGCCCTACAACTGGTACAAC | 5′ phosphate | + | MSDM |
| D34N | | | | |
| K71R | CCGACGGCGGCAGGTCCGGCG | 5′ phosphate | + | MSDM |
| G87S | CAAGAACAGCCGCTACGGCAGCGAC | 5′ phosphate | + | MSDM |
| G121D | CACATGAACCGCGACTACCCGGACAAG | 5′ phosphate | + | MSDM |
| G134R | CTGCCGGCCGGCCAGcGCTTCTGGCG | 5′ phosphate | + | MSDM |
| A141P | CGCAACGACTGCGCCGACCCGGG | 5′ phosphate | + | MSDM |
| I157L | GACGGTGACCGCTTCcTgGGCGGCGAGTCG | 5′ phosphate | + | MSDM |
| L178F, | CGCGACGAGTTTACCAACCTGCG | 5′ phosphate | + | MSDM |
| A179T | | | | |
| G223A | GGCGAGCTGTGGAAAgccCCTTCTGAATATCCG | 5′ phosphate | + | MSDM |
| H307L | gaacGGCGGCCAGCACctgTGGGCGCTGCAG | 5′ phosphate | + | MSDM |
| S334P, | GTACTGGccgCACATGTACGACTGGGGCTACGGC | 5′ phosphate | + | MSDM |
| D343E | gaaTTCATC | | | |

TABLE 7
Primer table for pPD77d20:

| | | | | |
|---|---|---|---|---|
| MUTATION | Oligo Sequence | MODIFICATION | Strand | PURPOSE |

| | | | | |
|---|---|---|---|---|
| N33Y, | GCGAAGCGGCCTACAACTGGTACAAC | 5′ phosphate | + | MSDM |
| D34N | | | | |
| K71R | CCGACGGCGGCAGGTCCGGCG | 5′ phosphate | + | MSDM |
| G121D | CACATGAACCGCGACTACCCGGACAAG | 5′ phosphate | + | MSDM |
| G134R | CTGCCGGCCGGCCAGcGCTTCTGGCG | 5′ phosphate | + | MSDM |
| A141P | CGCAACGACTGCGCCGACCCGGG | 5′ phosphate | + | MSDM |
| I157L | GACGGTGACCGCTTCcTgGGCGGCGAGTCG | 5′ phosphate | + | MSDM |
| L178F, | CGCGACGAGTTTACCAACCTGCG | 5′ phosphate | + | MSDM |
| A179T | | | | |
| G223A | GGCGAGCTGTGGAAAgccCCTTCTGAATATCCG | 5′ phosphate | + | MSDM |
| H307L | gaacGGCGGCCAGCACctgTGGGCGCTGCAG | 5′ phosphate | + | MSDM |
| S334P, | GTACTGGccgCACATGTACGACTGGGGCTAGGGC | 5′ phosphate | + | MSDM |
| D343E | gaaTTCATG | | | |

TABLE 8
Primer table for pPD77d34:

| | | | | |
|---|---|---|---|---|
| MUTATION | Oligo Sequence | MODIFICATION | Strand | PURPOSE |

| | | | | |
|---|---|---|---|---|
| N33Y, | GCGAAGCGCCCTACAACTGGTACAAC | 5′ phosphate | + | MSDM |
| D34N | | | | |
| G121D | CACATGAACCGCGACTACCCGGACAAG | 5′ phosphate | + | MSDM |
| G134R | CTGCCGGCCGGCCAGcGCTTCTGGCG | 5′ phosphate | + | MSDM |
| A141P | CGCAACGACTGCGCCGACCCGGG | 5′ phosphate | + | MSDM |
| I157L | GACGGTGACCGCTTCcTgGGCGGCGAGTCG | 5′ phosphate | + | MSDM |
| L178F, | CGCGACGAGTTTACCAACCTGCG | 5′ phosphate | + | MSDM |
| A179T | | | | |
| G223A | GGCGAGCTGTGGAAAgccCCTTCTGAATATCCG | 5′ phosphate | + | MSDM |
| H307L | gaacGGCGGCCAGCACctgTGGGCGCTGCAG | 5′ phosphate | + | MSDM |
| S334P | GTACTGGccgCACATGTACGACTGGGGCTACGGG | 5′ phosphate | + | MSDM |

Vector System Based on pPD77

The vector system used for pPD77 is based on pCRbluntTOPOII (invitrogen). The zeocin resistance cassette has been removed by pmlI, 393 bp fragment removed. The expression cassette from the pCC vector (P32-ssCGTase-PS4-tt) has then been inserted into the vector.

2. Ligation of PS4 Variant into pCCMini

The plasmid which contain the relevant mutations (created by MSDM) is cut with restriction enzyme Nco 1 and Hind III (Biolabs):
3 μg plasmid DNA, X μl 10× buffer 2, 10 units Nco1, 20 units HindIII, Incubation 2 h at 37° C.

Run digestion on a 1% agarose gel. Fragments sized 1293 bp (PS4 gene) is cut out of the gel and purified using Qiagen gel purification kit.

The vector pCCMini is then cut with restriction enzymes, Nco 1 and Hind III, and the digestion is then run on a 1% agarose gel. The fragment sized 3569 bp is cut out of the gel and purified using Qiagen gel purification kit.

Ligation: Use Rapid DNA ligation kit (Roche)

Use the double amount of insert compared to vector
e.g. 2 μl insert (PS4 gene)
   1 μl vector
   5 μl T4 DNA ligation buffer 2×conc
   1 μl dH₂O
   1 μl T4 DNA ligase

Ligate 5 min/RT

Transform the ligation into One Shot TOPO competent cells according to manufactures protocol (Invitrogen). Use 5 μl ligation pr. transformation.

Plate 50 μl transformations mix onto LB plates (33.6 g/l Lennox L Agar, Sigma) containing 1% starch and 0.05 μg/ml kanamycin. Vectors containing insert (PS4 variants) can be recognised by halo formation on the starch plates.

### Example 4

### Transformation into Bacillus subtilis (Protoplast Transformation)

Bacillus subtilis (strain DB104A; Smith et al. 1988; Gene 70, 351-361) is transformed with the mutated pCS-plasmids according to the following protocol.

A. Media for Protoplasting and Transformation
2×SMM per litre: 342 g sucrose (1 M); 4.72 g sodium maleate (0.04 M); 8:12 g MgCl₂, 6H₂O (0.04 M); pH 6.5 with concentrated NaOH. Distribute in 50-ml portions and autoclave for 10 min.
4×YT (½NaCl) 2 g Yeast extract+3.2 g Tryptone+0.5 g NaCl per 100 ml.
SMMP mix equal volumes of 2×SMM and 4×YT.
PEG 10 g polyethyleneglycol 6000 (BDH) or 8000 (Sigma) in 25 ml 1×SMM (autoclave for 10 min.).

B. Media for Plating/Regeneration
agar 4% Difco minimal agar. Autoclave for 15 min.
sodium succinate 270 g/l (1 M), pH 7.3 with HCl. Autoclave for 15 min.
phosphate buffer 3.5 g K₂HPO₄+1.5 g KH₂PO₄ per 100 ml. Autoclave for 15 min.
MgCl₂ 20.3 g MgCl₂, 6H₂O per 100 ml (1 M).
casamino acids 5% (w/v) solution. Autoclave for 15 min.
yeast extract 10 g per 100 ml, autoclave for 15 min.
glucose 20% (w/v) solution. Autoclave for 10 min.
DM3 regeneration medium: mix at 60 C (waterbath; 500-ml bottle):
   250 ml sodium succinate
   50 ml casamino acids
   25 ml yeast extract
   50 ml phosphate buffer
   15 ml glucose
   10 ml MgCl₂
   100 ml molten agar

Add appropriate antibiotics: chloramphenicol and tetracycline, 5 ug/ml; erythromycin, 1 ug/ml. Selection on kanamycin is problematic in DM3 medium: concentrations of 250 ug/ml may be required.

C. Preparation of Protoplasts

1. Use detergent-free plastic or glassware throughout.

2. Inoculate 10 ml of 2×YT medium in a 100-ml flask from a single colony. Grow an overnight culture at 25-30 C in a shaker (200 rev/min).

3. Dilute the overnight culture 20 fold into 100 ml of fresh 2×YT medium (250-ml flask) and grow until OD₆₀₀=0.4-0.5 (approx. 2 h) at 37 C in a shaker (200-250 rev/min).

4. Harvest the cells by centrifugation (9000 g, 20 min, 4 C).

5. Remove the supernatant with pipette and resuspend the cells in 5 ml of SMMP+5 mg lysozyme, sterile filtered.

6. Incubate at 37 C in a waterbath shaker (100 rev/min).

After 30 min and thereafter at 15 min intervals, examine 25 ul samples by microscopy. Continue incubation until 99% of the cells are protoplasted (globular appearance). Harvest the protoplasts by centrifugation (4000 g, 20 min, RT) and pipet off the supernatant. Resuspend the pellet gently in 1-2 ml of SMMP.

The protoplasts are now ready for use. (Portions (e.g. 0.15 ml) can be frozen at −80 C for future use (glycerol addition is not required). Although this may result in some reduction of transformability, 106 transformants per ug of DNA can be obtained with frozen protoplasts).

D. Transformation

1. Transfer 450 ul of PEG to a microtube.

2. Mix 1-10 ul of DNA (0.2 ug) with 150 ul of protoplasts and add the mixture to the microtube with PEG. Mix immediately, but gently.

3. Leave for 2 min at RT, and then add 1.5 ml of SMMP and mix.

4. Harvest protoplasts by microfuging (10 min, 13.000 rev/min (10-12.000 g)) and pour off the supernatant. Remove the remaining droplets with a tissue.

Add 300 ul of SMMP (do not vortex) and incubate for 60-90 min at 37 C in a waterbath shaker (100 rev/min) to allow for expression of antibiotic resistance markers. (The protoplasts become sufficiently resuspended through the shaking action of the waterbath.). Make appropriate dilutions in 1×SSM and plate 0.1 ml on DM3 plates

### Example 5

### Fermentation of PS4 Variants in Shake Flasks

The shake flask substrate is prepared as follows:

| Ingredient | % (w/v) |
|---|---|
| Water | — |
| Yeast extract | 2 |
| Soy Flour | 2 |
| NaCl | 0.5 |
| Dipotassium phosphate | 0.5 |
| Antifoam agent | 0.05 |

The substrate is adjusted to pH 6.8 with 4N sulfuric acid or sodium hydroxide before autoclaving. 100 ml of substrate is placed in a 500 ml flask with one baffle and autoclaved for 30 minutes. Subsequently, 6 ml of sterile dextrose syrup is added. The dextrose syrup is prepared by mixing one volume of 50% w/v dextrose with one volume of water followed by autoclaving for 20 minutes.

The shake flasks are inoculated with the variants and incubated for 24 hours at 35° C./180 rpm in an incubator. After incubation cells are separated from broth by centrifugation (10.000×g in 10 minutes) and finally, the supernatant is made cell free by microfiltration at 0.2 μm. The cell free supernatant is used for assays and application tests.

### Example 6

### Amylase Assays

Betamyl Assay

One Betamyl unit is defined as activity degrading 0,0351 mmole per 1 min. of PNP-coupled maltopentaose so that 0,0351 mmole PNP per 1 min. can be released by excess a-glucosidase in the assay mix. The assay mix contains 50 ul 50 mM Na-citrate, 5 mM CaCl₂, pH 6.5 with 25 ul enzyme sample and 25 ul Betamyl substrate (Glc5-PNP and a-glucosidase) from Megazyme, Ireland (1 vial dissolved in 10 ml water). The assay mix is incubated for 30 min. at 40 C and then stopped by adding 150 ul 4% Tris. Absorbance at 420 nm is measured using an ELISA-reader and the Betamyl activity is calculate based on Activity=A420*d in Betamyl units/ml of enzyme sample assayed.

Endo-Amylase Assay

The endo-amylase assay is identical to the Phadebas assay run according to manufacturer (Pharmacia & Upjohn Diagnostics AB).

II. Exo-Specificity

The ratio of exo-amylase activity to Phadebas activity was used to evaluate exo-specificity.

Specific Activity

For the PSac-D14, PSac-D20 and PSac-D34 variants we find an average specific activity of 10 Betamyl units per microgram of purified protein measured according to Bradford (1976; Anal. Biochem. 72, 248). This specific activity is used for based on activity to calculate the dosages used in the application trials.

### Example 7

### Half-Life Determination

t½ is defined as the time (in minutes) during which half the enzyme activity is inactivated under defined heat conditions. In order to determine the half life of the enzyme, the sample is heated for 1-10 minutes at constant temperatures of 60° C. to 90° C. The half life is calculated based on the residual Betamyl assay.

Procedure: In an Eppendorf vial, 1000 μl buffer is preheated for at least 10 minutes at 60° C. or higher. The heat treatment of the sample is started addition of 100 μl of the sample to the preheated buffer under continuous mixing (800 rpm) of the Eppendorf vial in an heat incubator (Termomixer comfort from Eppendorf). After 0, 2, 4, 6, 8 and 9 minutes of incubation, the treatment is stopped by transferring 45 μl of the sample to 1000 μl of the buffer equilibrated at 20° C. and incubating for one minute at 1500 rpm and at 20° C. The residual activity is measured with the Betamyl assay.

Calculation: Calculation of t½ is based on the slope of log10 (the base-10 logarithm) of the residual Betamyl activity versus the incubation time. t½ is calculated as Slope/0.301=t½.

### Example 8

### Results

TABLE 9
Biochemical properties of PSac-variants compared to wild-type PSac-cc1

| | | | Betamyl/ | |
|---|---|---|---|---|
| Variant | t½-75 | t½-80 | Phadebas | Mutations |
| PSac-cc1 | <0.5 | | 40 | |
| PSac-D3 | 9.3 | 3 | 43 | N33Y, D34N, K71R, G134R, A141P, |
| | | | | I157L, L178F, A179T, G223A, H307L, |
| | | | | D343E, S334P |
| PSac-D14 | 9.3 | 2.7 | 65 | N33Y, D34N, K71R, G87S, G121D, |
| (SEQ ID NO: 4) | | | | G134R, A141P, I157L, L178F, A179T, |
| | | | | G223A, H307L, D343E, S334P |
| PSac-D20 | 7.1 | 2.7 | 86 | N33Y, D34N, K71R, G121D, G134R, |
| (SEQ ID NO: 3) | | | | A141P, I157L, L178F, A179T, |
| | | | | G223A, H307L, D343E, S334P |
| PSac-D34 | 8.4 | 2.9 | 67 | N33Y, D34N, G121D, G134R, A141P, |
| (SEQ ID NO: 2) | | | | I157L, L178F, A179T, G223A, H307L, |
| | | | | S334P |
| PSac-pPD77d33 | 7.1 | 3 | 51 | N33Y, D34N, G134R, A141P, I157L, |
| (SEQ ID NO: 13) | | | | L178F, A179T, G223A, H307L, S334P |
| pMD55 | | 6.0 | 54 | N33Y D34N G121F G134R, A141P |
| | | | | I157L G223A H307L S334P L178F |
| | | | | A179T |
| pMD85 | | 5.1 | 115 | N33Y D34N G121F G134R, A141P |
| | | | | I157L G223E H307L S334P L178F |
| | | | | A179T |
| PMD96 | | 4.0 | 231 | N33Y D34N G121F G134R, A141P |
| | | | | I157L G223E H307L S334P L178F |
| | | | | A179T S161A |
| pMD86 | | 3.6 | 170 | N33Y D34N G121A G134R, A141P |
| | | | | I157L G223E H307L S334P L178F |
| | | | | A179T |
| pMD109 | | 3.6 | 170 | N33Y D34N G121A G134R, A141P |
| | | | | I157L G223E H307L S334P L178F |
| | | | | A179T S161A |

Sequences pPD77d40, pMD55, pMD85, pMD96, pMD86 and pMD109 have the residues at column 5 mutated and the starch binding domain deleted in a P. saccharophila wild type background (SEQ ID NO: 1). Their sequences may be constructed in a straightforward manner with this information.

### Example 9

### Model System Baking Tests

The doughs are made in the Farinograph at 30.0° C. 10.00 g reformed flour is weighed out and added in the Farinograph; after 1 min. mixing the reference/sample (reference=buffer or water, sample=enzyme+buffer or water) is added with a sterile pipette through the holes of the kneading vat. After 30 sec. the flour is scraped off the edges—also through the holes of the kneading vat. The sample is kneaded for 7 min.

A test with buffer or water is performed on the Farinograph before the final reference is run. FU should be 400 on the reference, if it is not, this should be adjusted with, for example, the quantity of liquid. The reference/sample is removed with a spatula and placed in the hand (with a disposable glove on it), before it is filled into small glass tubes (of approx. 4.5 cm's length) that are put in NMR tubes and corked up. 7 tubes per dough are made.

When all the samples have been prepared, the tubes are placed in a (programmable) water bath at 33° C. (without corks) for 25 min. and hereafter the water bath is set to stay for 5 min. at 33° C., then to heated to 98° C. over 56 min. (1.1° C. per minute) and finally to stay for 5 min. at 96° C.

The tubes are stored at 20.0° C. in a thermo cupboard. The solid content of the crumb was measured by proton NMR using a Bruker NMS 120 Minispec NMR analyser at day 1, 3 and 7 as shown for crumb samples prepared with 0, 05, 1 abnd 2 ppm PSacD34 in FIG. 2. The lower increase in solid content over time represents the reduction in amylopectin retrogradation.

After 7 days of storage at 20.0° C. in a thermo cupboard 10-20 mg samples of crumb weighed out and placed in 40 μl aluminium standard DSC capsules and kept at 20° C.

The capsules are used for Differential Scanning Calorimetry on a Mettler Toledo DSC 820 instrument. As parameters are used a heating cycle of 20-95° C. with 10° C. per min. heating and Gas/flow: N₂/80 ml per min. The results are analysed and the enthalpy for melting of retrograded amylopectin is calculated in J/g.

### Example 10

### Antistaling Effects

Model bread crumbs are prepared and measured according to Example 8. As shown in Table 2, PS4 variants show a strong reduction of the amylopectin retrogradation after baking as measured by Differential Scanning Calorimetry in comparison to the control. The PS4 variants shows a clear dosage effect.

### Example 11

### Firmness Effects in Baking Trials

Baking trials were carried out with a standard white bread sponge and dough recipe for US toast. The sponge dough is prepared from 1600 g of flour “All Purpose Classic” from Sisco Mills, USA”, 950 g of water, 40 g of soy bean oil and 32 g of dry yeast. The sponge is mixed for 1 min. at low speed and subsequently 3 min. at speed 2 on a Hobart spiral mixer. The sponge is subsequently fermented for 2.5 hours at 35° C., 85% RH followed by 0.5 hour at 5° C.

Thereafter 400 g of flour, 4 g of dry yeast, 40 g of salt, 2.4 g of calcium propionate, 240 g of high fructose corn sirup (Isosweet), 5 g of the emulsifier PANODAN 205, 5 g of enzyme active soy flour, 30 g of non-active soy flour, 220 g of water and 30 g of a solution of ascorbic acid (prepared from 4 g ascorbic acid solubilised in 500 g of water) are added to the sponge. The resulting dough is mixed for 1 min. at low speed and then 6 min. on speed 2 on a Diosna mixer. Thereafter the dough is rested for 5 min. at ambient temperature, and then 550 g dough pieces are scaled, rested for 5 min. and then sheeted on Glimek sheeter with the settings 1:4, 2:4, 3:15, 4:12 and 10 on each side and transferred to a baking form. After 60 min. proofing at 43° C. at 90% RH the doughs are baked for 29 min. at 218° C.

Firmness and resilience were measured with a TA-XT 2 texture analyser. The Softness, cohesiveness and resilience is determined by analysing bread slices by Texture Profile Analysis using a Texture Analyser From Stable Micro Systems, UK. The following settings were used:
Pre Test Speed: 2 mm/s
Test Speed: 2 mm/s
Post Test Speed: 10 mm/s
Rupture Test Distance: 1%
Distance: 40%
Force: 0.098 N
Time: 5.00 sec
Count: 5
Load Cell: 5 kg
Trigger Type: Auto—0.01 N

### Example 12

### Control of Volume of Danish Rolls

Danish Rolls are prepared from a dough based on 2000 g Danish reform flour (from Cerealia), 120 g compressed yeast, 32 g salt, and 32 g sucrose. Water is added to the dough according to prior water optimisation.

The dough is mixed on a Diosna mixer (2 min. at low speed and 5 min. at high speed). The dough temperature after mixing is kept at 26° C. 1350 g dough is scaled and rested for 10 min. in a heating cabinet at 30° C. The rolls are moulded on a Fortuna molder and proofed for 45 min. at 34° C. and at 85% relative humidity. Subsequently the rolls are baked in a Bago 2 oven for 18 min. at 250° C. with steam in the first 13 seconds. After baking the rolls are cooled for 25 min. before weighing and measuring of volume.

The rolls are evaluated regarding crust appearance, crumb homogeneity, capping of the crust, ausbund and specific volume (measuring the volume with the rape seed displacement method).

Based on these criteria it is found that the PS4 variants increase the specific volume and improve the quality parameters of Danish rolls. Thus PS4 variants are able to control the volume of baked products.

The invention will now be further described by the following numbered paragraphs:
1. A food additive comprising a PS4 variant polypeptide, in which the PS4 variant polypeptide is derivable from a parent polypeptide having non-maltogenic exoamylase activity, in which the PS4 variant polypeptide comprises an amino acid mutation at position 161 with reference to the position numbering of a Pseudomonas saccharophilia exoamylase sequence shown as SEQ ID NO: 1.
2. A food additive according to paragraph 1, in which the mutation at position 161 comprises a substitution 161A, preferably S161A.
3. A food additive according to paragraph 1 or 2, in which the PS4 variant polypeptide further comprises one or more further mutations at a position selected from the group consisting of: 121 and 223.
4. A food additive according to paragraph 3, in which the one or more further mutations is selected from the group consisting of: 121F, 121Y, 121W, 223E and 223K, more preferably G121F, G121Y, G121W, G223E and/or G223K.
5. A food additive according to any preceding paragraph, in which the PS4 variant polypeptide comprises mutations at positions selected from the group consisting of: 121, 161; 161, 223.
6. A food additive according to paragraph 5, in which the PS4 variant polypeptide comprises mutations at positions selected from the group consisting of: 121F/Y/W, 161A; 161A, 223E/K.
7. A food additive according to any preceding paragraph, in which the PS4 variant polypeptide comprises mutations at positions selected from the group consisting of: 121, 161 and 223.
8. A food additive according to any preceding paragraph, in which the PS4 variant polypeptide comprises mutations 121F/Y/W, 161A, 223E/K.
9. A food additive according to any preceding paragraph, in which the PS4 variant polypeptide further comprises one or mutations, preferably all, selected from the group consisting of positions: 134, 141, 157, 223, 307, 334.
10. A food additive according to any preceding paragraph, in which the PS4 variant polypeptide further comprises mutations at either or both positions 33 and 34.
11. A food additive according to paragraph 10, in which the PS4 variant polypeptide further comprises one or substitutions, preferably all, selected from the group consisting of: G134R, A141P, I157L, G223A, H307L, S334P, and optionally one or both of N33Y and D34N.
12. A food additive according to any preceding paragraph, in which the PS4 variant polypeptide further comprises:
   a. a mutation at position 121, preferably 121D, more preferably G121D;
   b. a mutation at position 178, preferably 178F, more preferably L178F;
   c. a mutation at position 179, preferably 179T, more preferably A179T; and/or
   d. a mutation at position 87, preferably 87S, more preferably G87S.
13. A food additive according to any preceding paragraph, in which the parent polypeptide comprises a non-maltogenic exoamylase, preferably a glucan 1,4-alpha-maltotetrahydrolase (EC 3.2.1.60).
14. A food additive according to any preceding paragraph, in which the parent polypeptide is or is derivable from Pseudomonas species, preferably Pseudomonas saccharophilia or Pseudomonas stutzeri.
15. A food additive according to any preceding paragraph, in which the parent polypeptide is a non-maltogenic exoamylase from Pseudomonas saccharophilia exoamylase having a sequence shown as SEQ ID NO: 1 or SEQ ID NO: 5.
16. A food additive according to any of paragraphs 1 to 14, in which the parent polypeptide is a non-maltogenic exoamylase from Pseudomonas stutzeri having a sequence shown as SEQ ID NO: 7 or SEQ ID NO: 11.
17. A food additive according to any preceding paragraph, which comprises a sequence as set out in the description, claims or figures.
18. A food additive according to any preceding paragraph, in which the PS4 variant polypeptide has a higher thermostability compared to the parent polypeptide or a wild type polypeptide when tested under the same conditions.
19. A food additive according to any preceding paragraph, in which the half life (t½), preferably at 60 degrees C., is increased by 15% or more, preferably 50% or more, most preferably 100% or more, relative to the parent polypeptide or the wild type polypeptide.
20. A food additive according to any preceding paragraph, in which the PS4 variant polypeptide has a higher exo-specificity compared to the parent polypeptide or a wild type polypeptide when tested under the same conditions.
21. A food additive according to any preceding paragraph, in which the PS4 variant polypeptide has 10% or more, preferably 20% or more, preferably 50% or more, exo-specificity compared to the parent polypeptide or the wild type polypeptide.
22. Use of a PS4 variant polypeptide as set out in any preceding paragraph as a food additive.
23. A process for treating a starch comprising contacting the starch with a PS4 variant polypeptide as set out in any of paragraphs 1 to 21 and allowing the polypeptide to generate from the starch one or more linear products.
24. Use of a PS4 variant polypeptide as set out in any of paragraphs 1 to 21 in preparing a food product.
25. A process of preparing a food product comprising admixing a polypeptide as set out in any of paragraphs 1 to 21 with a food ingredient.
26. Use according to paragraph 24, or a process according to paragraph 25, in which the food product comprises a dough or a dough product, preferably a processed dough product.
27. A use or process according to any of paragraphs 24 to 26, in which the food product is a bakery product.
28. A process for making a bakery product comprising: (a) providing a starch medium; (b) adding to the starch medium a PS4 variant polypeptide as set out in any of paragraphs 1 to 21; and (c) applying heat to the starch medium during or after step (b) to produce a bakery product.
29. A food product, dough product or a bakery product obtained by a process according to any of paragraphs 24 to 28.
30. An improver composition for a dough, in which the improver composition comprises a PS4 variant polypeptide as set out in any of paragraphs 1 to 21, and at least one further dough ingredient or dough additive.
31. A composition comprising a flour and a PS4 variant polypeptide as set out in any of paragraphs 1 to 21.
32. Use of a PS4 variant polypeptide as set out in any of paragraphs 1 to 21, in a dough product to retard or reduce staling, preferably detrimental retrogradation, of the dough product.
33. A combination of a PS4 variant polypeptide as set out in any preceding paragraph, together with Novamyl, or a variant, homologue, or mutants thereof which has maltogenic alpha-amylase activity.
34. Use of a combination according to paragraph 33 for an application according to any preceding paragraph.
35. A food product produced by treatment with a combination according to paragraph 34.
36. Use of a PS4 variant polypeptide substantially as hereinbefore described with reference to and as shown in the accompanying drawings.
37. A combination comprising a PS4 nucleic acid substantially as hereinbefore described with reference to and as shown in the accompanying drawings.

### REFERENCES

Penninga, D., van der Veen, B. A., Knegtel, R. M., van Hijum, S. A., Rozeboom, H. J., Kalk, K. H., Dijkstra, B. W., Dijkhuizen, L. (1996). The raw starch binding domain of cyclodextrin glycosyltransferase from Bacillus circulans strain 251. J. Biol. Chem. 271, 32777-32784.
Sambrook J, F.E.M.T. (1989). Molecular Cloning: A Laboratory Manual, 2nd Edn. Cold Spring Harbor Laboratory, Cold Spring Harbor N.Y.
Zhou, J. H., Baba, T., Takano, T., Kobayashi, S., Arai, Y. (1989). Nucleotide sequence of the maltotetraohydrolase gene from Pseudomonas saccharophila. FEBS Lett. 255, 37-41.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

Sequence Listings

SEQ ID NO: 1

PS4 reference sequence, derived from Pseudomonas saccharophila maltotetrahydrolase amino acid sequence.

| | |
|---|---|
| 1 | DQAGKSPAGV RYHGGDEIIL QGFHWNVVRE AP [symbol] WYNILR QQASTIAADG FSAIWMPVPW |
| 61 | RDFSSWTDGG KSGGGEGYFW HDFNKNGRYG SDAQLRQAAG ALGGAGVKVL YDVVPNHMNR |
| 121 | [symbol] YPDKEINLP AGQ [symbol] FWRNDC [symbol] DPGNYPNDC DDGDRF [symbol] GGE SDLNTGHPQI YGMFRDE [symbol] [symbol] N |
| 181 | LRSGYGAGGF RFDFVRGYAP ERVDSWMSDS ADSSFCVGEL WK [symbol] PSEYPSW DWRNTASWQQ |
| 241 | IIKDWSDRAK CPVFDFALKE RMQNGSVADW KHGLNGNPDP RWREVAVTFV DNHDTGYSPG |
| 301 | QNGGQH [symbol] WAL QDGLIRQAYA YILTSPGTPV VYW [symbol] HMYDWG YGDFIRQLIQ VRRTAGVRAD |
| 361 | SAISFHSGYS GLVATVSGSQ QTLVVALNSD LANPGQVASG SFSEAVNASN GQVRVWRSGS |
| 421 | GDGGGNDGGE GGLVNVNFRC DNGVTQMGDS VYAVGNVSQL GNWSPASAVR LTDTSSYPTW |
| 481 | KGSIALPDGQ NVEWKCLIRN EADATLVRQW QSGGNNQVQA AAGASTSGSF |

SEQ ID NO: 2

PSac-D34 sequence; Pseudomonas saccharophila maltotetrahydrolase amino acid sequence with 11 substitutions and deletion of the starch binding domain.

| | |
|---|---|
| 1 | DQAGKSPAGV RYHGGDEIIL QGFHWNVVRE AP [symbol] WYNILR QQASTIAADG FSAIWMPVPW |
| 61 | RDFSSWTDPG KSGGGEGYFW HDFNKNGRYG SDAQLRQAAG ALGGAGVKVL YDVVPNHMNR |
| 121 | [symbol] YPDKETNLP AGQ [symbol] FWRNDC [symbol] DPGNYPNDC DDGDRF [symbol] GGE SDLNTGHPQI YGMFRDE [symbol] N |
| 181 | LRSGYGAGGF RFDFVRGYAP ERVDSWMSDS ADSSFCVGEL WK [symbol] PSEYPSW DWRNTASWQQ |
| 241 | IIKDWSDRAK CPVFDFALKE RMQNGSVADW KHGLNGNPDP RWREVAVTFV DNHDTGYSPG |
| 301 | QNGGQH [symbol] WAL QDGLIRQAYA YILTSPGTPV VYW [symbol] HMYDWG YGDFIRQLIQ VRRTAGVRAD |
| 361 | SAISFHSGYS GLVATVSGSQ QTLVVALNSD LANPGQVASG SFSEAVNASN GQVRVWRSGS |
| 421 | GDGGGNDGG |

SEQ ID NO: 3

PSac-D20 sequence; Pseudomonas saccharophila maltotetrahydrolase amino acid sequence with 13 substitutions and deletion of the starch binding domain.

| | |
|---|---|
| 1 | DQAGKSPAGV RYHGGDEIIL QGFHWNVVRE AP [symbol] WYNILR QQASTIAADG FSAIWMPVPW |
| 61 | RDFSSWTDPG [symbol] SGGGEGYFW HDFNKNGRYG SDAQLRQAAG ALGGAGVKVL YDVVPNHMNR |
| 121 | [symbol] YPDKEINLP AGQ [symbol] FWRNDC [symbol] DPGNYPNDC DDGDRF [symbol] GGE SDLNTGHPQI YGMFRDE [symbol] N |
| 181 | LRSGYGAGGF RFDFVRGYAP ERVDSWMSDS ADSSFCVGEL WK [symbol] PSEYPSW DWRNTASWQQ |
| 241 | IIKDWSDRAK CPVFDFALKE RMQNGSVADW KHGLNGNPDP RWREVAVTFV DNHDTGYSPG |
| 301 | QNGGQH [symbol] WAL QDCLIRQAYA YILTSPGTPV VYW [symbol] HMYDWG YG [symbol] FIRQLIQ VRRTAGVRAD |
| 361 | SAISFHSGYS GLVATVSGSQ QTLVVALNSD LANPGQVASG SFSEAVNASN GQVRVWRSGS |
| 421 | GDGGGNDGG |

SEQ ID NO: 4

PSac-D14 sequence; Pseudomonas saccharophila maltotetrahydrolase amino acid sequence with 14 substitutions and deletion of the starch binding domain.

| | |
|---|---|
| 1 | DQAGKSPAGV RYHGGDEIIL QGFHWNVVRE AP [symbol] WYNILR QQASTIAADG FSAIWMPVPW |
| 61 | RDFSSWTDPG [symbol] SGGGEGYFW HDFNKN [symbol] RYG SDAQLRQAAG ALGGAGVKVL YDVVPNHMNR |
| 121 | DYPDKEINLP AGQ [symbol] FWRNDC [symbol] DPGNYPNDC DDGDRF [symbol] GGE SDLNTGHPQI YGMFRDE [symbol] N |
| 181 | LRSGYGAGGF RFDFVRGYAP ERVDSWMSDS ADSSFCVGEL WK [symbol] PSEYPSW DWRNTASWQQ |
| 241 | IIKDWSDRAK CPVFDFALKE RMQNGSVADW KHGLNGNPDP RWREVAVTFV DNHDTGYSPG |
| 301 | QNGGQHLWAL QDCLIRQAYA YILTSPGTPV VYW [symbol] HMYDWG YG [symbol] FIRQLIQ VRRTAGVRAD |
| 361 | SAISFHSGYS GLVATVSGSQ QTLVVALNSD LANPGQVASG SFSEAVNASN GQVRVWRSGS |
| 421 | GDGGGNDGG |

SEQ ID NO: 5

Pseudomonas saccharophila Glucan 1,4-alpha-maltotetrahydrolase precursor (EC 3.2.1.60) (G4-amylase) (Maltotetraose-forming amylase) (Exo-maltotetraohydrolase) (Maltotetraose-forming exo-amylase). SWISS-PROT accession number P22963.

MSHILRAAVL AAVLLPFPAL ADQAGKSPAG VRYHGGDEII LQGFHWNVVR EAPNDWYNIL
RQQASTIAAD GFSAIWMPVP WRDFSSWTDG GKSGGGEGYF WHDFNKNGRY GSDAQLRQAA
GALGGAGVKV LYDVVPNHMN RGYPDKEINL PAGQGFWRND CADPGNYPND CDDGDRFIGG
ESDLNTGHPQ IYGMFRDELA NLRSGYGAGG FRFDFVRGYA PERVDSWMSD SADSSFCVGE
LWKGPSEYPS WDWRNTASWQ QIIKDWSDRA KCPVFDFALK ERMQNGSVAD WKHGLNGNPD
PRWREVAVTF VDNHDTGYSP GQNGGQHHWA LQDGLIRQAY AYILTSPGTP VVYWSHMYDW
GYGDFIRQLI QVRRTAGVRA DSAISFHSGY SGLVATVSGS QQTLVVALNS DLANPGQVAS
GSFSEAVNAS NGQVRVWRSG SGDGGGNDGG EGGLVNVNFR CDNGVTQMGD SVYAVGNVSQ
LGNWSPASAV RLTDTSSYPT WKGSIALPDG QNVEWKCLIR NEADATLVRQ WQSGGNNQVQ
AAAGASTSGS F

SEQ ID NO: 6

P. saccharophila mta gene encoding maltotetraohydrolase (EC number=3.2.1.60). GenBank accession number X16732.

gatcggcgta ggtttcgcat tcgttgccca ggcgatattt cgccggtgcg ccagcagcct
ggaagcaggc ctggtcgccg ccgccggccg tggcgccgac gcccgaacgc agatagccgt
ggaaatcgac cgccagggcc gggccgccga ccagcagggc ggcaagcagg caggcgggtt
ttaggacgaa cagggggtgc gcggtgtgct tcatgacgag gtccttgttt ttcttgttaa
tgccgaatcg atcacgcctt cgctgcgtgt cgcagggcgc agctcggtgg cgaaagcctc
ggggatggct ccgctggcgg catcctcccg accagagatt tcgctggcgc agctcgaggg
cgtaatcagg atgagtgcgg cgtaatccct ggggtggggc tacgcccggc agggcgcaga
tgattgccag gggccttcgg cctggccact acgccgcctg caactgggcg ggggaggttg
gtggtcgggg cgtgcagggg cagcctgcgg gtgccggtcg aagacccggc cggcgttcat
cctcgtccgg cggccttgcc gtaggatacc cgaacaagca caagaaccgg agtattgcga
tgagccacat cctgcgtgcc gccgtattgg cggcggtcct gctgccgttt cccgcactgg
ccgatcaggc cggcaagagc ccggccgggg tgcgctacca cggcggcgac gaaatcatcc
tccagggctt ccactggaac gtcgtccgcg aagcgcccaa cgactggtac aacatcctcc
gccaacaggc ctcgacgatc gcggccgacg gcttctcggc aatctggatg ccggtgccct
ggcgtgactt ctccagctgg accgacggcg gcaagtccgg cggcggcgaa ggctacttct
ggcacgactt caacaagaac ggccgctacg gcagcgacgc ccagctgcgc caggccgccg
gcgcactcgg tggcgccggg gtgaaggtgc tctacgatgt ggtgcccaat cacatgaacc
gcggctaccc ggacaaggag atcaacctgc cggccggcca gggcttctgg cgcaacgact
gcgccgaccc gggcaactac cccaacgact gcgacgacgg tgaccgcttc atcggcggcg
agtcggacct gaacaccggc catccgcaga tttacggcat gtttcgcgac gagcttgcca
acctgcgcag cggctacggc gccggcggct tccgcttcga cttcgttcgc ggctatgcgc
ccgagcgggt cgacagctgg atgagcgaca gcgccgacag cagcttctgc gttggcgagc
tgtggaaagg cccttctgaa tatccgagct gggactggcg caacacggcg agctggcagc
agatcatcaa ggactggtcc gaccgggcca agtgcccggt gttcgacttc gctctcaagg
agcgcatgca gaacggctcg gtcgccgact ggaagcatgg cctcaatggc aaccccgacc
cgcgctggcg cgaggtggcg gtgaccttcg tcgacaacca cgacaccggc tattcgcccg
ggcagaacgg cggccagcac cactgggcgc tgcaggacgg gctgatccgc caggcctacg
cctacatcct caccagcccg ggcacgccgg tggtgtactg gtcgcacatg tacgactggg
gctacggcga cttcatccgc cagctgatcc aggtgcggcg caccgccggc gtgcgcgccg
attcggcgat cagcttccat agcggctaca gcggtctggt cgctaccgtc agcggcagcc
agcagaccct ggtggtggcg ctcaactccg atctggccaa ccccggccag gttgccagcg
gcagcttcag cgaggcggtc aacgccagca acggccaggt gcgcgtctgg cgcagcggta
gcggcgatgg cggcgggaat gacggcggcg agggtggctt ggtcaatgtg aactttcgct
gcgacaacgg cgtgacgcag atgggcgaca gcgtctacgc ggtgggcaac gtcagccagc
tcggcaactg gagcccggcc tccgcggtac ggctgaccga caccagcagc tatccgacct
ggaagggcag catcgccctg cctgacggtc agaacgtgga atggaagtgc ctgatccgca
acgaggcgga cgcgacgctg gtgcgtcagt ggcaatcggg cggcaacaac caggtccagg
ccgccgccgg cgcgagcacc agcggctcgt tctgacgaca tgcccgcccg gcctcggcta
cgcctacgcc gggcggctcc tcccgaccca gggtgggcag ggaggaggcc ggcgacgggc
cgggccgccg atgctggcac gacaaccata aaagccttcg cgctgcgctg tcgtatcagg
agctgttcat gttggcccag acccgctcga cccctttccg gcttggcttc ctggcccggc
tgtacctgct gatcgccgca ctggtggcct tgctgatgct ggtagccggc accagcctgg
ttgccatcgg ccgcctgcaa ggcaatgccg agcaaatctc gtcgaccgcg tcgcgtctgc
tggtcagcga gagcttcttc ggtacgttgc agagcctgac gcagaacctg tccgacgccc
tggccgagga ccggcctgac cagctcgacg gctatgtcgg ccggcatcgc acgctgcagg
accaggccct cgagctgttc gcccagctgg agcgggtgac gccggcacat gccgagacca
agcaagcctg gcggcgctgt tgccggagct cgaccgccgc agcctggcgc tgatcgatgc
gcacgcgacc tgctcgcgcg tggggcgcaa cgccgtcgcc tgcgcgatct gcagctgcag
ttctcgcggc tcaagcagga cctgctgcag gcgcagttcg tgacgggcga cgagctggtc
gcctattcca tcaagcagtt catcatcccg ctcgagcagg tcgagcgctg ctgttcgatg
ccatcggcgt gtcttcgatc aaggcactcg atgaagcggg tgcgcagatc

SEQ ID NO:7

PS4 reference sequence, derived from Pseudomonas stutzeri maltotetrahydrolase amino acid sequence.

| | |
|---|---|
| 1 | DQAGKSPNAV RYHGGDEIIL QGFHWNVVRE AP [symbol] WYNILR QQAATIAADG FSAIWMPVPW |
| 61 | RDFSSWSDGS KSGGGEGYFW HDFNKN [symbol] RYG SDAQLRQAAS ALGGAGVKVL YDVVPNHMNR |
| 121 | [symbol] YPDKEINLP AGQ [symbol] FWRNDC [symbol] DPGNYPNDC DDGDRF [symbol] GGD ADLNTGHPQV YGMFRDEFTN |
| 181 | LRSQYGAGGF RFDFVRGYAP ERVNSWMTDS ADNSFCVGEL WK [symbol] PSEYPNW DWRNTASWQQ |
| 241 | IIKDWSDRAK CPVFDFALKE RMQNGSIADW KHGLNGNPDP RWREVAVTFV DNHDTGYSPG |
| 301 | QNGGQH [symbol] WAL QDGLIRQAYA YILTSPGTPV VYW [symbol] HMYDWG YGDFIRQLIQ VRRAAGVRAD |
| 361 | SAISFHSGYS GLVATVSGSQ QTLVVALNSD LGNPGQVASG SFSEAVNASN GQVRVWRSGT |
| 421 | GSGGGEPGAL VSVSFRCDNG ATQMGDSVYA VGNVSQLGNW SPAAALRLTD TSGYPTWKGS |
| 481 | IALPAGQNEE WKCLIRNEAN ATQVRQWQGG ANNSLTPSEG ATTVGRL |

SEQ ID NO: 8

PStu-D34 sequence; Pseudomonas stutzeri maltotetrahydrolase amino acid sequence with 9 substitutions.

| | |
|---|---|
| 1 | DQAGKSPNAV RYHGGDEIIL QGFHWNVVRE AP [symbol] WYNILR QQAATIAADG FSAIWMPVPW |
| 61 | RDFSSWSDPS KSGGGEGYFW HDFNKNGRYG SDAQLRQAAS ALGGAGVKVL YDVVPNHMNR |
| 121 | [symbol] YPDKEINLP AGQ [symbol] FWRNDC [symbol] DPGNYPNDC DDGDRF [symbol] GGD ADLNTGHPQV YGMFRDEFTN |
| 181 | LRSQYGAGGF RFDFVRGYAP ERVNSWMTDS ADNSFCVGEL WK [symbol] PSEYPNW DWRNTASWQQ |
| 241 | IIKDWSDRAK CPVFDFALKE RMQNGSIADW KHGLNGNPDP RWREVAVTFV DNHDTGYSPG |
| 301 | QNGGQH [symbol] WAL QDGLIRQAYA YILTSPGTPV VYW [symbol] HMYDWG YGDFIRQLIQ VRRAAGVRAD |
| 361 | SAISFHSGYS GLVATVSGSQ QTLVVALNSD LGNPGQVASG SFSEAVNASN GQVRVWRSGT |
| 421 | GSGGGEPGAL VSVSFRCDNG ATQMGDSVYA VGNVSQLGNW SPAAALRLTD TSGYPTWKGS |
| 481 | IALPAGQNEE WKCLIRNEAN ATQVRQWQGG ANNSLTPSEG ATTVGRL |

SEQ ID NO: 9

PStu-D20 sequence; Pseudomonas stutzeri maltotetrahydrolase amino acid sequence with 11 substitutions.

| | |
|---|---|
| 1 | DQAGKSPNAV RYHGGDEIIL QGFHWNVVRE AP [symbol] WYNILR QQAATIAADG FSAIWMPVPW |
| 61 | RDFSSWSDPS [symbol] SGGGEGYFW HDFNKNGRYG SDAQLRQAAS ALGGAGVKVL YDVVPNHMNR |
| 121 | [symbol] YPDKEINLP AGQ [symbol] FWRNDC [symbol] DPGNYPNDC DDGDRF [symbol] GGD ADLNTGHPQV YGMFRDEFTN |
| 181 | LRSQYGAGGF RFDFVRGYAP ERVNSWMTDS ADNSFCVGEL WK [symbol] PSEYPNW DWRNTASWQQ |
| 241 | IIKDWSDRAK CPVFDFALKE RMQNGSIADW KHGLNGNPDP RWREVAVTFV DNHDTGYSPG |
| 301 | QNGGQH [symbol] WAL QDGLIRQAYA YILTSPGTPV VYW [symbol] HMYDWG YG [symbol] FIRQLIQ VRRAAGVRAD |
| 361 | SAISFHSGYS GLVATVSGSQ QTLVVALNSD LGNPGQVASG SFSEAVNASN GQVRVWRSGT |
| 421 | GSGGGEPGAL VSVSFRCDNG ATQMGDSVYA VGNVSQLGNW SPAAALRLTD TSGYPTWKGS |
| 481 | IALPAGQNEE WKCLIRNEAN ATQVRQWQGG ANNSLTPSEG ATTVGRL |

SEQ ID NO: 10

PStu-D14 sequence; Pseudomonas stutzeri maltotetrahydrolase amino acid sequence with 12 substitutions.

| | |
|---|---|
| 1 | DQAGKSPNAV RYHGGDEIIL QGFHWNVVRE AP [symbol] WYNILR QQAATIAADG FSAIWMPVPW |
| 61 | RDFSSWSDPS [symbol] SGGGEGYFW HDFNKN [symbol] RYG SDAQLRQAAS ALGGAGVKVL YDVVPNHMNR |
| 121 | [symbol] YPDKEINLP AGQ [symbol] FWRNDC [symbol] DPGNYPNDC DDGDRF [symbol] GGD ADLNTGHPQV YGMFRDEFTN |
| 181 | LRSQYGAGGF RFDFVRGYAP ERVNSWMTDS ADNSFCVGEL WK [symbol] PSEYPNW DWRNTASWQQ |
| 241 | IIKDWSDRAK CPVFDFALKE RMQNGSIADW KHGLNGNPDP RWREVAVTFV DNHDTGYSPG |
| 301 | QNGGQH [symbol] WAL QDGLIRQAYA YILTSPGTPV VYW [symbol] HMYDWG YG [symbol] FIRQLIQ VRRAAGVRAD |
| 361 | SAISFHSGYS GLVATVSGSQ QTLVVALNSD LGNPGQVASG SFSEAVNASN GQVRVWRSGT |
| 421 | GSGGGEPGAL VSVSFRCDNG ATQMGDSVYA VGNVSQLGNW SPAAALRLTD TSGYPTWKGS |
| 481 | IALPAGQNEE WKCLIRNEAN ATQVRQWQGG ANNSLTPSEG ATTVGRL |

SEQ ID NO: 11

Pseudomonas stutzeri (Pseudomonas perfectomarina). Glucan 1,4-alpha-maltotetrahydrolase precursor (EC 3.2.1.60) (G4-amylase) (Maltotetraose-forming amylase) (Exo-maltotetraohydrolase) (Maltotetraose-forming exo-amylase). SWISS-PROT accession number P13507.

MSHILRAAVL AANLLPLPSM ADQAGKSPNA VRYHGGDEII LQGFHWNVVR EAPNDWYNIL
RQQAATIAAD GFSAIWMPVP WRDFSSWSDG SKSGGGEGYF WHDFNKNGRY GSDAQLRQAA
SALGGAGVKV LYDVVPNHMN RGYPDKEINL PAGQGFWRND CADPGNYPND CDDGDRFIGG
DADLNTGHPQ VYGMFRDEFT NLRSQYGAGG FRFDFVRGYA PERVNSWMTD SADMSFCVGE
LWKGPSEYPN WDWRNTASWQ QIIKDWSDPA KCPVFDFALK ERMQNGSIAD WKHGLNGNPD
PRWREVAVTF VDNHDTGYSP GQNGGQHHWA LQDGLIRQAY AYILTSPGTP VVYWSHMYDW
GYGDFIRQLI QVRRAAGVRA DSAISFHSGY SGLVATVSGS QQTLVVALNS DLGNPGQVAS
GSFSEAVNAS NGQVRVWRSG TGSGGGEPGA LVSVSFRCDN GATQMGDSVY AVGNVSQLGN
WSPAAALRLT DTSGYPTWKG SIALPAGQNE EWKCLIRNEA NATQVRQWQG GANNSLTPSE
GATTVGRL

SEQ ID NO: 12

P. stutzeri maltotetraose-forming amylase (amyP) gene, complete cds. GenBank accession number M24516.

| | |
|---|---|
| 1 | gatcggcctt tacggaaagt gatagagctt ctcttccggc aaactttgtt ccccagtgac |
| 61 | agagggttag tatcggatcg cttcctcttt gggtttggta gatcaggagc gccgagagca |
| 121 | ggatgaaatc ctgcggccag aaggtcgcgc cgaagatgtg gaactgctgc tggccgagat |
| 181 | ccggccggcg ttcatcctcg tccggcggcc ttgccgccag ctacccgaac aagcacaaga |
| 241 | accggagtat tgcgatgagc cacatcctgc gagccgccgt attggcggcg atgctgttgc |
| 301 | cgttgccgtc catggccgat caggccggca agagccccaa cgctgtgcgc taccacggcg |
| 361 | gcgacgaaat cattctccag ggctttcact ggaacgtcgt ccgcgaagcg cccaacgact |
| 421 | ggtacaacat cctgcgccag caggccgcga ccatcgccgc cgacggcttc tcggcgatct |
| 481 | ggatgccggt gccctggcgc gacttctcca gctggagcga cggcagcaag tccggcggcg |
| 541 | gtgaaggcta cttctggcac gacttcaaca agaacggccg ctatggcagt gacgcccagc |
| 601 | tgcgtcaggc cgccagcgcg ctcggtggcg ccggcgtgaa agtgctttac gacgtggtgc |
| 661 | ccaaccacat gaaccgtggc tatccggaca aggagatcaa cctcccggcc ggccagggct |
| 721 | tctggcgcaa cgactgcgcc gacccgggca actaccccaa tgattgcgac gacggcgacc |
| 781 | gcttcatcgg cggcgatgcg gacctcaaca ccggccaccc gcaggtctac ggcatgttcc |
| 841 | gcgatgaatt caccaacctg cgcagtcagt acggtgccgg cggcttccgc ttcgactttg |
| 901 | ttcggggcta tgcgccggag cgggtcaaca gctggatgac cgatagcgcc gacaacagct |
| 961 | tctgcgtcgg cgaactgtgg aaaggcccct ctgagtaccc gaactgggac tggcgcaaca |
| 1021 | ccgccagctg gcagcagatc atcaaggact ggtccgaccg ggccaagtgc ccggtgttcg |
| 1081 | acttcgccct caaggaacgc atgcagaacg ctcgatcgcc gactggaagc acgcctgaac |
| 1141 | ggcaatcccg acccgcgtgg cgcgaggtgg cggtgacctt cgtcgacaac cacgacaccg |
| 1201 | gctactcgcc cgggcagaac ggtgggcagc accactgggc tctgcaggac gggctgatcc |
| 1261 | gccaggccta cgcctacatc ctcaccagcc ccggtacgcc ggtggtgtac tggtcgcaca |
| 1321 | tgtacgactg gggttacggc gacttcatcc gtcagctgat ccaggtgcgt cgcgccgccg |
| 1381 | gcgtgcgcgc cgattcggcg atcagcttcc acagcggcta cagcggtctg gtcgccaccg |
| 1441 | tcagcggcag ccagcagacc ctggtggtgg cgctcaactc cgacctgggc aatcccggcc |
| 1501 | aggtggccag cggcagcttc agcgaggcgg tcaacgccag caacggccag gtgcgcgtgt |
| 1561 | ggcgtagcgg cacgggcagc ggtggcggtg aacccggcgc tctggtcagt gtgagtttcc |
| 1621 | gctgcgacaa cggcgcgacg cagatgggcg acagcgtcta cgcggtcggc aacgtcagcc |
| 1681 | agctcggtaa ctggagcccg gccgcggcgt tgcgcctgac cgacaccagc ggctacccga |
| 1741 | cctggaaggg cagcattgcc ttgcctgccg gccagaacga ggaatggaaa tgcctgatcc |
| 1801 | gcaacgaggc caacgccacc caggtgcggc aatggcaggg cggggcaaac aacagcctga |
| 1861 | cgccgagcga gggcgccacc accgtcggcc ggctctagcc cgggcggcaa ctcggccgtc |
| 1921 | tcgcggatgt gaggcggctg gtctcggcgg cggtatcgtt gcgctggggg cggggccgcc |
| 1981 | gttcacgcgc cctgctatcg ctagttttcg gcgctccgcg catcggccag ttgccagcga |
| 2041 | atcgcctgcg cttcggcctg gtgcaggtcg tcgagcagcg ct |

SEQ ID NO: 13

pMD55 sequence; Pseudomonas saccharophila maltotetrahydrolase amino acid sequence with 11 substitutions (G134R, A141P, I157L, G223A, H307L, S334P, N33Y, D34N, L178F, A179T and G121F) and deletion of the starch binding domain.

| | |
|---|---|
| 1 | DQAGKSPNAV RYHGGDEIIL QGFHWNVVRE AP [symbol] WYNILR QQASTIAADG FSAIWMPVPW |
| 61 | RDFSSWSDPS KSGGGEGYFW HDFNKNGRYG SDAQLRQAAG ALGGAGVKVL YDVVPNHMNR |
| 121 | [symbol] YPDKEINLP AGQ [symbol] RFWRNDC [symbol] DPGNYPNDC DDGDRF [symbol] GGE SDLNTGHPQI YGMFRDE [symbol] N |
| 181 | LRSQYGAGGF RFDFVRGYAP ERVNSWMTDS ADNSFCVGEL WK [symbol] PSEYPNW DWRNTASWQQ |
| 241 | IIKDWSDRAK CPVFDFALKE RMQNGSIADW KHGLNGNPDP RWREVAVTFV DNHDTGYSPG |
| 301 | QNGGQH [symbol] WAL QDGLIRQAYA YILTSPGTPV VYW [symbol] HMYDWG YGDFIRQLIQ VRRAAGVRAD |
| 361 | SAISFHSGYS GLVATVSGSQ QTLVVALNSD LGNPGQVASG SFSEAVNASN GQVRVWRSGT |
| 421 | GDGGGNDGG |

1. A food additive comprising a PS4 variant polypeptide, in which the PS4 variant polypeptide is derivable from a parent polypeptide having non-maltogenic exoamylase activity, in which the PS4 variant polypeptide comprises an amino acid mutation at position 161 with reference to the position numbering of a Pseudomonas saccharophilia exoamylase sequence shown as SEQ ID NO: 1.

2. A food additive according to claim 1, in which the mutation at position 161 comprises a substitution 161A.

3. A food additive according to claim 1, in which the PS4 variant polypeptide further comprises one or more further mutations at a position selected from the group consisting of: 121 and 223.

4. A food additive according to claim 3, in which the one or more further mutations is selected from the group consisting of: 121F, 121Y, 121W, 223E and 223K.

5. A food additive according to claim 1, in which the PS4 variant polypeptide comprises one or more mutations at positions selected from the group consisting of: 121, 161; 161, and 223.

6. A food additive according to claim 5, in which the PS4 variant polypeptide comprises one or more mutations at positions selected from the group consisting of: 121F/Y/W, 161A, 161A, and 223E/K.

7. A food additive according to claim 1, in which the PS4 variant polypeptide comprises one or more mutations at positions selected from the group consisting of: 121, 161 and 223.

8. A food additive according to claim 1, in which the PS4 variant polypeptide comprises mutations 121F/Y/W, 161A, and 223E/K.

9. A food additive according to claim 1, in which the PS4 variant polypeptide further comprises one or more mutations, selected from the group consisting of positions: 134, 141, 157, 223, 307, and 334.

10. A food additive according to claim 1, in which the PS4 variant polypeptide further comprises mutations at either or both of positions 33 and 34.

11. A food additive according to claim 10, in which the PS4 variant polypeptide further comprises one or more substitutions, selected from the group consisting of: G134R, A141P, I157L, G223A, H307L, and S334P, and optionally one or both of N33Y and D34N.

12. A food additive according to claim 1, in which the PS4 variant polypeptide further comprises:
a. a mutation at position 121;
b. a mutation at position 178;
c. a mutation at position 179; and/or
d. a mutation at position 87.

13. A food additive according to claim 1, in which the parent polypeptide comprises a non-maltogenic exoamylase.

14. A food additive according to claim 1, in which the parent polypeptide is or is derivable from Pseudomonas species, preferably Pseudomonas saccharophilia or Pseudomonas stutzeri.

15. A food additive according to claim 1, in which the parent polypeptide is a non-maltogenic exoamylase from Pseudomonas saccharophilia exoamylase having a sequence shown as SEQ ID NO: 1 or SEQ ID NO: 5.

16. A food additive according to claim 1, in which the parent polypeptide is a non-maltogenic exoamylase from Pseudomonas stutzeri having a sequence shown as SEQ ID NO: 7 or SEQ ID NO: 11.

17. A food additive according to claim 1, which comprises a sequence as set out in the description, claims or figures.

18. A food additive according to claim 1, in which the PS4 variant polypeptide has a higher thermostability compared to the parent polypeptide or a wild type polypeptide when tested under the same conditions.

19. A food additive according to claim 1, in which the half life (t½), at 60 degrees C., is increased by 15% or more, 50% or more, or 100% or more, relative to the parent polypeptide or the wild type polypeptide.

20. A food additive according to claim 1, in which the PS4 variant polypeptide has a higher exo-specificity compared to the parent polypeptide or a wild type polypeptide when tested under the same conditions.

21. A food additive according to claim 1, in which the PS4 variant polypeptide has 10% or more, 20% or more, or 50% or more, exo-specificity compared to the parent polypeptide or the wild type polypeptide.

22. A method of using a PS4 variant polypeptide as set out in claim 1 as a food additive.

23. A process for treating a starch comprising contacting the starch with a PS4 variant polypeptide as set out in claim 1 and allowing the polypeptide to generate from the starch one or more linear products.

24. A method of using a PS4 variant polypeptide as set out in claim 1 in preparing a food product.

25. A process of preparing a food product comprising admixing a polypeptide as set out in claim 1 with a food ingredient.

26. The method of claim 24, in which the food product comprises a dough or a dough product, preferably a processed dough product.

27. A method of claim 24, in which the food product is a bakery product.

28. A process for making a bakery product comprising: (a) providing a starch medium; (b) adding to the starch medium a PS4 variant polypeptide as set out in claim 1; and (c) applying heat to the starch medium during or after step (b) to produce a bakery product.

29. A food product, dough product or a bakery product obtained by a method according to claim 24.

30. An improver composition for a dough, in which the improver composition comprises a PS4 variant polypeptide as set out in claim 1, and at least one further dough ingredient or dough additive.

31. A composition comprising a flour and a PS4 variant polypeptide as set out in claim 1.

32. A method of using a PS4 variant polypeptide as set out in claim 1, in a dough product to retard or reduce staling, preferably detrimental retrogradation, of the dough product.

33. A combination of a PS4 variant polypeptide as set out in claim 1, together with Novamyl, or a variant, homologue, or mutants thereof which has maltogenic alpha-amylase activity.

34. (canceled)

35. A food product produced by treatment with a combination according to claim 33.

36. (canceled)

37. (canceled)

38. A food additive according to claim 4, in which the one or more further mutations is selected from the group consisting of: G121F, G121Y, G121W, G223E and G223K.

39. A process according to claim 25, in which the food product comprises a dough or a dough product, preferably a processed dough product.

40. A process according to claim 25, in which the food product is a bakery product.

41. A food product, dough product or a bakery product obtained by a process according to claim 25.

42. A food product, dough product or a bakery product obtained by a process according to claim 28.


SEQUENCE LISTING (63 sequences)

SEQ ID NO: 1 - protein, 530 aa, Pseudomonas saccharophila
DQAGKSPAGVRYHGGDEIILQGFHWNVVREAPNDWYNILRQQASTIAADGFSAIWMPVPWRDFSSWTDGGKSGGGEGYFWHDFNKNGRYGSDAQLRQAAGALGGAGVKVLYDVVPNHMNRGYPDKEINLPAGQGFWRNDCADPGNYPNDCDDGDRFIGGESDLNTGHPQIYGMFRDELANLRSGYGAGGFRFDFVRGYAPERVDSWMSDSADSSFCVGELWKGPSEYPSWDWRNTASWQQIIKDWSDRAKCPVFDFALKERMQNGSVADWKHGLNGNPDPRWREVAVTFVDNHDTGYSPGQNGGQHHWALQDGLIRQAYAYILTSPGTPVVYWSHMYDWGYGDFIRQLIQVRRTAGVRADSAISFHSGYSGLVATVSGSQQTLVVALNSDLANPGQVASGSFSEAVNASNGQVRVWRSGSGDGGGNDGGEGGLVNVNFRCDNGVTQMGDSVYAVGNVSQLGNWSPASAVRLTDTSSYPTWKGSIALPDGQNVEWKCLIRNEADATLVRQWQSGGNNQVQAAAGASTSGSF

SEQ ID NO: 2 - protein, 429 aa, Pseudomonas saccharophila
DQAGKSPAGVRYHGGDEIILQGFHWNVVREAPYNWYNILRQQASTIAADGFSAIWMPVPWRDFSSWTDPGKSGGGEGYFWHDFNKNGRYGSDAQLRQAAGALGGAGVKVLYDVVPNHMNRDYPDKEINLPAGQRFWRNDCPDPGNYPNDCDDGDRFLGGESDLNTGHPQIYGMFRDEFTNLRSGYGAGGFRFDFVRGYAPERVDSWMSDSADSSFCVGELWKAPSEYPSWDWRNTASWQQIIKDWSDRAKCPVFDFALKERMQNGSVADWKHGLNGNPDPRWREVAVTFVDNHDTGYSPGQNGGQHLWALQDGLIRQAYAYILTSPGTPVVYWPHMYDWGYGDFIRQLIQVRRTAGVRADSAISFHSGYSGLVATVSGSQQTLVVALNSDLANPGQVASGSFSEAVNASNGQVRVWRSGSGDGGGNDGG

SEQ ID NO: 3 - protein, 429 aa, Pseudomonas saccharophila
DQAGKSPAGVRYHGGDEIILQGFHWNVVREAPYNWYNILRQQASTIAADGFSAIWMPVPWRDFSSWTDPGRSGGGEGYFWHDFNKNGRYGSDAQLRQAAGALGGAGVKVLYDVVPNHMNRDYPDKEINLPAGQRFWRNDCPDPGNYPNDCDDGDRFLGGESDLNTGHPQIYGMFRDEFTNLRSGYGAGGFRFDFVRGYAPERVDSWMSDSADSSFCVGELWKAPSEYPSWDWRNTASWQQIIKDWSDRAKCPVFDFALKERMQNGSVADWKHGLNGNPDPRWREVAVTFVDNHDTGYSPGQNGGQHLWALQDGLIRQAYAYILTSPGTPVVYWPHMYDWGYGEFIRQLIQVRRTAGVRADSAISFHSGYSGLVATVSGSQQTLVVALNSDLANPGQVASGSFSEAVNASNGQVRVWRSGSGDGGGNDGG

SEQ ID NO: 4 - protein, 429 aa, Pseudomonas saccharophila
DQAGKSPAGVRYHGGDEIILQGFHWNVVREAPYNWYNILRQQASTIAADGFSAIWMPVPWRDFSSWTDPGRSGGGEGYFWHDFNKNSRYGSDAQLRQAAGALGGAGVKVLYDVVPNHMNRDYPDKEINLPAGQRFWRNDCPDPGNYPNDCDDGDRFLGGESDLNTGHPQIYGMFRDEFTNLRSGYGAGGFRFDFVRGYAPERVDSWMSDSADSSFCVGELWKAPSEYPSWDWRNTASWQQIIKDWSDRAKCPVFDFALKERMQNGSVADWKHGLNGNPDPRWREVAVTFVDNHDTGYSPGQNGGQHLWALQDGLIRQAYAYILTSPGTPVVYWPHMYDWGYGEFIRQLIQVRRTAGVRADSAISFHSGYSGLVATVSGSQQTLVVALNSDLANPGQVASGSFSEAVNASNGQVRVWRSGSGDGGGNDGG

SEQ ID NO: 5 - protein, 551 aa, Pseudomonas saccharophila
MSHILRAAVLAAVLLPFPALADQAGKSPAGVRYHGGDEIILQGFHWNVVREAPNDWYNILRQQASTIAADGFSAIWMPVPWRDFSSWTDGGKSGGGEGYFWHDFNKNGRYGSDAQLRQAAGALGGAGVKVLYDVVPNHMNRGYPDKEINLPAGQGFWRNDCADPGNYPNDCDDGDRFIGGESDLNTGHPQIYGMFRDELANLRSGYGAGGFRFDFVRGYAPERVDSWMSDSADSSFCVGELWKGPSEYPSWDWRNTASWQQIIKDWSDRAKCPVFDFALKERMQNGSVADWKHGLNGNPDPRWREVAVTFVDNHDTGYSPGQNGGQHHWALQDGLIRQAYAYILTSPGTPVVYWSHMYDWGYGDFIRQLIQVRRTAGVRADSAISFHSGYSGLVATVSGSQQTLVVALNSDLANPGQVASGSFSEAVNASNGQVRVWRSGSGDGGGNDGGEGGLVNVNFRCDNGVTQMGDSVYAVGNVSQLGNWSPASAVRLTDTSSYPTWKGSIALPDGQNVEWKCLIRNEADATLVRQWQSGGNNQVQAAAGASTSGSF

SEQ ID NO: 6 - DNA, 3050 bp, Pseudomonas saccharophila
gatcggcgtaggtttcgcattcgttgcccaggcgatatttcgccggtgcgccagcagcctggaagcaggcctggtcgccgccgccggccgtggcgccgacgcccgaacgcagatagccgtggaaatcgaccgccagggccgggccgccgaccagcagggcggcaagcaggcaggcgggttttaggacgaacagggggtgcgcggtgtgcttcatgacgaggtccttgtttttcttgttaatgccgaatcgatcacgccttcgctgcgtgtcgcagggcgcagctcggtggcgaaagcctcggggatggctccgctggcggcatcctcccgaccagagatttcgctggcgcagctcgagggcgtaatcaggatgagtgcggcgtaatccctggggtggggctacgcccggcagggcgcagatgattgccaggggccttcggcctggccactacgccgcctgcaactgggcgggggaggttggtggtcggggcgtgcaggggcagcctgcgggtgccggtcgaagacccggccggcgttcatcctcgtccggcggccttgccgtaggatacccgaacaagcacaagaaccggagtattgcgatgagccacatcctgcgtgccgccgtattggcggcggtcctgctgccgtttcccgcactggccgatcaggccggcaagagcccggccggggtgcgctaccacggcggcgacgaaatcatcctccagggcttccactggaacgtcgtccgcgaagcgcccaacgactggtacaacatcctccgccaacaggcctcgacgatcgcggccgacggcttctcggcaatctggatgccggtgccctggcgtgacttctccagctggaccgacggcggcaagtccggcggcggcgaaggctacttctggcacgacttcaacaagaacggccgctacggcagcgacgcccagctgcgccaggccgccggcgcactcggtggcgccggggtgaaggtgctctacgatgtggtgcccaatcacatgaaccgcggctacccggacaaggagatcaacctgccggccggccagggcttctggcgcaacgactgcgccgacccgggcaactaccccaacgactgcgacgacggtgaccgcttcatcggcggcgagtcggacctgaacaccggccatccgcagatttacggcatgtttcgcgacgagcttgccaacctgcgcagcggctacggcgccggcggcttccgcttcgacttcgttcgcggctatgcgcccgagcgggtcgacagctggatgagcgacagcgccgacagcagcttctgcgttggcgagctgtggaaaggcccttctgaatatccgagctgggactggcgcaacacggcgagctggcagcagatcatcaaggactggtccgaccgggccaagtgcccggtgttcgacttcgctctcaaggagcgcatgcagaacggctcggtcgccgactggaagcatggcctcaatggcaaccccgacccgcgctggcgcgaggtggcggtgaccttcgtcgacaaccacgacaccggctattcgcccgggcagaacggcggccagcaccactgggcgctgcaggacgggctgatccgccaggcctacgcctacatcctcaccagcccgggcacgccggtggtgtactggtcgcacatgtacgactggggctacggcgacttcatccgccagctgatccaggtgcggcgcaccgccggcgtgcgcgccgattcggcgatcagcttccatagcggctacagcggtctggtcgctaccgtcagcggcagccagcagaccctggtggtggcgctcaactccgatctggccaaccccggccaggttgccagcggcagcttcagcgaggcggtcaacgccagcaacggccaggtgcgcgtctggcgcagcggtagcggcgatggcggcgggaatgacggcggcgagggtggcttggtcaatgtgaactttcgctgcgacaacggcgtgacgcagatgggcgacagcgtctacgcggtgggcaacgtcagccagctcggcaactggagcccggcctccgcggtacggctgaccgacaccagcagctatccgacctggaagggcagcatcgccctgcctgacggtcagaacgtggaatggaagtgcctgatccgcaacgaggcggacgcgacgctggtgcgtcagtggcaatcgggcggcaacaaccaggtccaggccgccgccggcgcgagcaccagcggctcgttctgacgacatgcccgcccggcctcggctacgcctacgccgggcggctcctcccgacccagggtgggcagggaggaggccggcgacgggccgggccgccgatgctggcacgacaaccataaaagccttcgcgctgcgctgtcgtatcaggagctgttcatgttggcccagacccgctcgacccctttccggcttggcttcctggcccggctgtacctgctgatcgccgcactggtggccttgctgatgctggtagccggcaccagcctggttgccatcggccgcctgcaaggcaatgccgagcaaatctcgtcgaccgcgtcgcgtctgctggtcagcgagagcttcttcggtacgttgcagagcctgacgcagaacctgtccgacgccctggccgaggaccggcctgaccagctcgacggctatgtcggccggcatcgcacgctgcaggaccaggccctcgagctgttcgcccagctggagcgggtgacgccggcacatgccgagaccaagcaagcctggcggcgctgttgccggagctcgaccgccgcagcctggcgctgatcgatgcgcacgcgacctgctcgcgcgtggggcgcaacgccgtcgcctgcgcgatctgcagctgcagttctcgcggctcaagcaggacctgctgcaggcgcagttcgtgacgggcgacgagctggtcgcctattccatcaagcagttcatcatcccgctcgagcaggtcgagcgctgctgttcgatgccatcggcgtgtcttcgatcaaggcactcgatgaagcgggtgcgcagatc

SEQ ID NO: 7 - protein, 527 aa, Pseudomonas saccharophila
DQAGKSPNAVRYHGGDEIILQGFHWNVVREAPNDWYNILRQQAATIAADGFSAIWMPVPWRDFSSWSDGSKSGGGEGYFWHDFNKNGRYGSDAQLRQAASALGGAGVKVLYDVVPNHMNRGYPDKEINLPAGQGFWRNDCADPGNYPNDCDDGDRFIGGDADLNTGHPQVYGMFRDEFTNLRSQYGAGGFRFDFVRGYAPERVNSWMTDSADNSFCVGELWKGPSEYPNWDWRNTASWQQIIKDWSDRAKCPVFDFALKERMQNGSIADWKHGLNGNPDPRWREVAVTFVDNHDTGYSPGQNGGQHHWALQDGLIRQAYAYILTSPGTPVVYWSHMYDWGYGDFIRQLIQVRRAAGVRADSAISFHSGYSGLVATVSGSQQTLVVALNSDLGNPGQVASGSFSEAVNASNGQVRVWRSGTGSGGGEPGALVSVSFRCDNGATQMGDSVYAVGNVSQLGNWSPAAALRLTDTSGYPTWKGSIALPAGQNEEWKCLIRNEANATQVRQWQGGANNSLTPSEGATTVGRL

SEQ ID NO: 8 - protein, 527 aa, Pseudomonas saccharophila
DQAGKSPNAVRYHGGDEIILQGFHWNVVREAPYNWYNILRQQAATIAADGFSAIWMPVPWRDFSSWSDPSKSGGGEGYFWHDFNKNGRYGSDAQLRQAASALGGAGVKVLYDVVPNHMNRDYPDKEINLPAGQRFWRNDCPDPGNYPNDCDDGDRFLGGDADLNTGHPQVYGMFRDEFTNLRSQYGAGGFRFDFVRGYAPERVNSWMTDSADNSFCVGELWKAPSEYPNWDWRNTASWQQIIKDWSDRAKCPVFDFALKERMQNGSIADWKHGLNGNPDPRWREVAVTFVDNHDTGYSPGQNGGQHLWALQDGLIRQAYAYILTSPGTPVVYWPHMYDWGYGDFIRQLIQVRRAAGVRADSAISFHSGYSGLVATVSGSQQTLVVALNSDLGNPGQVASGSFSEAVNASNGQVRVWRSGTGSGGGEPGALVSVSFRCDNGATQMGDSVYAVGNVSQLGNWSPAAALRLTDTSGYPTWKGSIALPAGQNEEWKCLIRNEANATQVRQWQGGANNSLTPSEGATTVGRL

SEQ ID NO: 9 - protein, 527 aa, Pseudomonas saccharophila
DQAGKSPNAVRYHGGDEIILQGFHWNVVREAPYNWYNILRQQAATIAADGFSAIWMPVPWRDFSSWSDPSRSGGGEGYFWHDFNKNGRYGSDAQLRQAASALGGAGVKVLYDVVPNHMNRDYPDKEINLPAGQRFWRNDCPDPGNYPNDCDDGDRFLGGDADLNTGHPQVYGMFRDEFTNLRSQYGAGGFRFDFVRGYAPERVNSWMTDSADNSFCVGELWKAPSEYPNWDWRNTASWQQIIKDWSDRAKCPVFDFALKERMQNGSIADWKHGLNGNPDPRWREVAVTFVDNHDTGYSPGQNGGQHLWALQDGLIRQAYAYILTSPGTPVVYWPHMYDWGYGEFIRQLIQVRRAAGVRADSAISFHSGYSGLVATVSGSQQTLVVALNSDLGNPGQVASGSFSEAVNASNGQVRVWRSGTGSGGGEPGALVSVSFRCDNGATQMGDSVYAVGNVSQLGNWSPAAALRLTDTSGYPTWKGSIALPAGQNEEWKCLIRNEANATQVRQWQGGANNSLTPSEGATTVGRL

SEQ ID NO: 10 - protein, 527 aa, Pseudomonas saccharophila
DQAGKSPNAVRYHGGDEIILQGFHWNVVREAPYNWYNILRQQAATIAADGFSAIWMPVPWRDFSSWSDPSRSGGGEGYFWHDFNKNSRYGSDAQLRQAASALGGAGVKVLYDVVPNHMNRDYPDKEINLPAGQRFWRNDCPDPGNYPNDCDDGDRFLGGDADLNTGHPQVYGMFRDEFTNLRSQYGAGGFRFDFVRGYAPERVNSWMTDSADNSFCVGELWKAPSEYPNWDWRNTASWQQIIKDWSDRAKCPVFDFALKERMQNGSIADWKHGLNGNPDPRWREVAVTFVDNHDTGYSPGQNGGQHLWALQDGLIRQAYAYILTSPGTPVVYWPHMYDWGYGEFIRQLIQVRRAAGVRADSAISFHSGYSGLVATVSGSQQTLVVALNSDLGNPGQVASGSFSEAVNASNGQVRVWRSGTGSGGGEPGALVSVSFRCDNGATQMGDSVYAVGNVSQLGNWSPAAALRLTDTSGYPTWKGSIALPAGQNEEWKCLIRNEANATQVRQWQGGANNSLTPSEGATTVGRL

SEQ ID NO: 11 - protein, 548 aa, Pseudomonas saccharophila
MSHILRAAVLAAMLLPLPSMADQAGKSPNAVRYHGGDEIILQGFHWNVVREAPNDWYNILRQQAATIAADGFSAIWMPVPWRDFSSWSDGSKSGGGEGYFWHDFNKNGRYGSDAQLRQAASALGGAGVKVLYDVVPNHMNRGYPDKEINLPAGQGFWRNDCADPGNYPNDCDDGDRFIGGDADLNTGHPQVYGMFRDEFTNLRSQYGAGGFRFDFVRGYAPERVNSWMTDSADNSFCVGELWKGPSEYPNWDWRNTASWQQIIKDWSDRAKCPVFDFALKERMQNGSIADWKHGLNGNPDPRWREVAVTFVDNHDTGYSPGQNGGQHHWALQDGLIRQAYAYILTSPGTPVVYWSHMYDWGYGDFIRQLIQVRRAAGVRADSAISFHSGYSGLVATVSGSQQTLVVALNSDLGNPGQVASGSFSEAVNASNGQVRVWRSGTGSGGGEPGALVSVSFRCDNGATQMGDSVYAVGNVSQLGNWSPAAALRLTDTSGYPTWKGSIALPAGQNEEWKCLIRNEANATQVRQWQGGANNSLTPSEGATTVGRL

SEQ ID NO: 12 - DNA, 2082 bp, Pseudomonas saccharophila
gatcggcctttacggaaagtgatagagcttctcttccggcaaactttgttccccagtgacagagggttagtatcggatcgcttcctctttgggtttggtagatcaggagcgccgagagcaggatgaaatcctgcggccagaaggtcgcgccgaagatgtggaactgctgctggccgagatccggccggcgttcatcctcgtccggcggccttgccgccagctacccgaacaagcacaagaaccggagtattgcgatgagccacatcctgcgagccgccgtattggcggcgatgctgttgccgttgccgtccatggccgatcaggccggcaagagccccaacgctgtgcgctaccacggcggcgacgaaatcattctccagggctttcactggaacgtcgtccgcgaagcgcccaacgactggtacaacatcctgcgccagcaggccgcgaccatcgccgccgacggcttctcggcgatctggatgccggtgccctggcgcgacttctccagctggagcgacggcagcaagtccggcggcggtgaaggctacttctggcacgacttcaacaagaacggccgctatggcagtgacgcccagctgcgtcaggccgccagcgcgctcggtggcgccggcgtgaaagtgctttacgacgtggtgcccaaccacatgaaccgtggctatccggacaaggagatcaacctcccggccggccagggcttctggcgcaacgactgcgccgacccgggcaactaccccaatgattgcgacgacggcgaccgcttcatcggcggcgatgcggacctcaacaccggccacccgcaggtctacggcatgttccgcgatgaattcaccaacctgcgcagtcagtacggtgccggcggcttccgcttcgactttgttcggggctatgcgccggagcgggtcaacagctggatgaccgatagcgccgacaacagcttctgcgtcggcgaactgtggaaaggcccctctgagtacccgaactgggactggcgcaacaccgccagctggcagcagatcatcaaggactggtccgaccgggccaagtgcccggtgttcgacttcgccctcaaggaacgcatgcagaacgctcgatcgccgactggaagcacgcctgaacggcaatcccgacccgcgtggcgcgaggtggcggtgaccttcgtcgacaaccacgacaccggctactcgcccgggcagaacggtgggcagcaccactgggctctgcaggacgggctgatccgccaggcctacgcctacatcctcaccagccccggtacgccggtggtgtactggtcgcacatgtacgactggggttacggcgacttcatccgtcagctgatccaggtgcgtcgcgccgccggcgtgcgcgccgattcggcgatcagcttccacagcggctacagcggtctggtcgccaccgtcagcggcagccagcagaccctggtggtggcgctcaactccgacctgggcaatcccggccaggtggccagcggcagcttcagcgaggcggtcaacgccagcaacggccaggtgcgcgtgtggcgtagcggcacgggcagcggtggcggtgaacccggcgctctggtcagtgtgagtttccgctgcgacaacggcgcgacgcagatgggcgacagcgtctacgcggtcggcaacgtcagccagctcggtaactggagcccggccgcggcgttgcgcctgaccgacaccagcggctacccgacctggaagggcagcattgccttgcctgccggccagaacgaggaatggaaatgcctgatccgcaacgaggccaacgccacccaggtgcggcaatggcagggcggggcaaacaacagcctgacgccgagcgagggcgccaccaccgtcggccggctctagcccgggcggcaactcggccgtctcgcggatgtgaggcggctggtctcggcggcggtatcgttgcgctgggggcggggccgccgttcacgcgccctgctatcgctagttttcggcgctccgcgcatcggccagttgccagcgaatcgcctgcgcttcggcctggtgcaggtcgtcgagcagcgct

SEQ ID NO: 13 - protein, 429 aa, Pseudomonas saccharophila
DQAGKSPAGVRYHGGDEIILQGFHWNVVREAPYNWYNILRQQASTIAADGFSAIWMPVPWRDFSSWTDPGKSGGGEGYFWHDFNKNGRYGSDAQLRQAAGALGGAGVKVLYDVVPNHMNRFYPDKEINLPAGQRFWRNDCPDPGNYPNDCDDGDRFLGGESDLNTGHPQIYGMFRDEFTNLRSGYGAGGFRFDFVRGYAPERVDSWMSDSADSSFCVGELWKAPSEYPSWDWRNTASWQQIIKDWSDRAKCPVFDFALKERMQNGSVADWKHGLNGNPDPRWREVAVTFVDNHDTGYSPGQNGGQHLWALQDGLIRQAYAYILTSPGTPVVYWPHMYDWGYGDFIRQLIQVRRTAGVRADSAISFHSGYSGLVATVSGSQQTLVVALNSDLANPGQVASGSFSEAVNASNGQVRVWRSGSGDGGGNDGG

SEQ ID NO: 14 - protein, 21 aa, Pseudomonas saccharophila
MSHILRAAVLAAVLLPFPALA

SEQ ID NO: 15 - DNA, 21 bp, Artificial Sequence
  Description of Artificial Sequence Primer
atgacgaggtccttgtttttc

SEQ ID NO: 16 - DNA, 18 bp, Artificial Sequence
  Description of Artificial Sequence Primer
cgctagtcgtccatgtcg

SEQ ID NO: 17 - DNA, 27 bp, Artificial Sequence
  Description of Artificial Sequence Primer
gccatggatcaggccggcaagagcccg

SEQ ID NO: 18 - DNA, 25 bp, Artificial Sequence
  Description of Artificial Sequence Primer
tggatcctcagaacgagccgctggt

SEQ ID NO: 19 - DNA, 25 bp, Artificial Sequence
  Description of Artificial Sequence Primer
gaattcagccgccgtcattcccgcc

SEQ ID NO: 20 - DNA, 20 bp, Artificial Sequence
  Description of Artificial Sequence Primer
agatttacggcatgtttcgc

SEQ ID NO: 21 - DNA, 20 bp, Artificial Sequence
  Description of Artificial Sequence Primer
tagccgctatggaagctgat

SEQ ID NO: 22 - DNA, 20 bp, Artificial Sequence
  Description of Artificial Sequence Primer
tgaccttcgtcgacaaccac

SEQ ID NO: 23 - DNA, 20 bp, Artificial Sequence
  Description of Artificial Sequence Primer
gatagctgctggtgacggtc

SEQ ID NO: 24 - DNA, 26 bp, Artificial Sequence
  Description of Artificial Sequence Primer
ctgccggccggccagcgcttctggcg

SEQ ID NO: 25 - DNA, 26 bp, Artificial Sequence
  Description of Artificial Sequence Primer
cgccagaagcgctggccggccggcag

SEQ ID NO: 26 - DNA, 30 bp, Artificial Sequence
  Description of Artificial Sequence Primer
gacggtgaccgcttcctgggcggcgagtcg

SEQ ID NO: 27 - DNA, 30 bp, Artificial Sequence
  Description of Artificial Sequence Primer
cgactcgccgcccaggaagcggtcaccgtc

SEQ ID NO: 28 - DNA, 33 bp, Artificial Sequence
  Description of Artificial Sequence Primer
ggcgagctgtggaaagccccttctgaatatccg

SEQ ID NO: 29 - DNA, 33 bp, Artificial Sequence
  Description of Artificial Sequence Primer
cggatattcagaaggggctttccacagctcgcc

SEQ ID NO: 30 - DNA, 31 bp, Artificial Sequence
  Description of Artificial Sequence Primer
gaacggcggccagcacctgtgggcgctgcag

SEQ ID NO: 31 - DNA, 31 bp, Artificial Sequence
  Description of Artificial Sequence Primer
ctgcagcgcccacaggtgctggccgccgttc

SEQ ID NO: 32 - DNA, 43 bp, Artificial Sequence
  Description of Artificial Sequence Primer
gtactggccgcacatgtacgactggggctacggcgaattcatc

SEQ ID NO: 33 - DNA, 43 bp, Artificial Sequence
  Description of Artificial Sequence Primer
gatgaattcgccgtagccccagtcgtacatgtgcggccagtac

SEQ ID NO: 34 - DNA, 26 bp, Artificial Sequence
  Description of Artificial Sequence Primer
gcgaagcgccctacaactggtacaac

SEQ ID NO: 35 - DNA, 21 bp, Artificial Sequence
  Description of Artificial Sequence Primer
ccgacggcggcaggtccggcg

SEQ ID NO: 36 - DNA, 25 bp, Artificial Sequence
  Description of Artificial Sequence Primer
caagaacagccgctacggcagcgac

SEQ ID NO: 37 - DNA, 27 bp, Artificial Sequence
  Description of Artificial Sequence Primer
cacatgaaccgcgactacccggacaag

SEQ ID NO: 38 - DNA, 26 bp, Artificial Sequence
  Description of Artificial Sequence Primer
ctgccggccggccagcgcttctggcg

SEQ ID NO: 39 - DNA, 23 bp, Artificial Sequence
  Description of Artificial Sequence Primer
cgcaacgactgcgccgacccggg

SEQ ID NO: 40 - DNA, 30 bp, Artificial Sequence; identical to SEQ ID NO: 26
  Description of Artificial Sequence Primer

SEQ ID NO: 41 - DNA, 23 bp, Artificial Sequence
  Description of Artificial Sequence Primer
cgcgacgagtttaccaacctgcg

SEQ ID NO: 42 - DNA, 33 bp, Artificial Sequence; identical to SEQ ID NO: 28
  Description of Artificial Sequence Primer

SEQ ID NO: 43 - DNA, 31 bp, Artificial Sequence; identical to SEQ ID NO: 30
  Description of Artificial Sequence Primer

SEQ ID NO: 44 - DNA, 43 bp, Artificial Sequence; identical to SEQ ID NO: 32
  Description of Artificial Sequence Primer

SEQ ID NO: 45 - DNA, 26 bp, Artificial Sequence
  Description of Artificial Sequence Primer
gcgaagcgccctacaactggtacaac

SEQ ID NO: 46 - DNA, 21 bp, Artificial Sequence
  Description of Artificial Sequence Primer
ccgacggcggcaggtccggcg

SEQ ID NO: 47 - DNA, 27 bp, Artificial Sequence
  Description of Artificial Sequence Primer
cacatgaaccgcgactacccggacaag

SEQ ID NO: 48 - DNA, 26 bp, Artificial Sequence
  Description of Artificial Sequence Primer
ctgccggccggccagcgcttctggcg

SEQ ID NO: 49 - DNA, 23 bp, Artificial Sequence
  Description of Artificial Sequence Primer
cgcaacgactgcgccgacccggg

SEQ ID NO: 50 - DNA, 30 bp, Artificial Sequence; identical to SEQ ID NO: 26
  Description of Artificial Sequence Primer

SEQ ID NO: 51 - DNA, 23 bp, Artificial Sequence
  Description of Artificial Sequence Primer
cgcgacgagtttaccaacctgcg

SEQ ID NO: 52 - DNA, 33 bp, Artificial Sequence; identical to SEQ ID NO: 28
  Description of Artificial Sequence Primer

SEQ ID NO: 53 - DNA, 31 bp, Artificial Sequence; identical to SEQ ID NO: 30
  Description of Artificial Sequence Primer

SEQ ID NO: 54 - DNA, 43 bp, Artificial Sequence; identical to SEQ ID NO: 32
  Description of Artificial Sequence Primer

SEQ ID NO: 55 - DNA, 26 bp, Artificial Sequence
  Description of Artificial Sequence Primer
gcgaagcgccctacaactggtacaac

SEQ ID NO: 56 - DNA, 27 bp, Artificial Sequence
  Description of Artificial Sequence Primer
cacatgaaccgcgactacccggacaag

SEQ ID NO: 57 - DNA, 26 bp, Artificial Sequence
  Description of Artificial Sequence Primer
ctgccggccggccagcgcttctggcg

SEQ ID NO: 58 - DNA, 23 bp, Artificial Sequence
  Description of Artificial Sequence Primer
cgcaacgactgcgccgacccggg

SEQ ID NO: 59 - DNA, 30 bp, Artificial Sequence; identical to SEQ ID NO: 26
  Description of Artificial Sequence Primer

SEQ ID NO: 60 - DNA, 23 bp, Artificial Sequence
  Description of Artificial Sequence Primer
cgcgacgagtttaccaacctgcg

SEQ ID NO: 61 - DNA, 33 bp, Artificial Sequence; identical to SEQ ID NO: 28
  Description of Artificial Sequence Primer

SEQ ID NO: 62 - DNA, 31 bp, Artificial Sequence; identical to SEQ ID NO: 30
  Description of Artificial Sequence Primer

SEQ ID NO: 63 - DNA, 34 bp, Artificial Sequence
  Description of Artificial Sequence Primer
gtactggccgcacatgtacgactggggctacggc